(19) United States
(12) Patent Application Publication
(10) Pub. No.: US 2023/0071507 A1
(43) Pub. Date: Mar. 9, 2023

(54) MACROPHAGE-DERIVED ENGINEERED VESICLES FOR TARGETED DELIVERY AND TREATMENT

(71) Applicant: University of Kentucky Research Foundation, Lexington, KY (US)

(72) Inventors: Chris Richards, Versailles, KY (US); Jill Kolesar, Lexington, KY (US)

(21) Appl. No.: 17/799,082

(22) PCT Filed: Feb. 11, 2021
(86) PCT No.: PCT/US21/17720

§ 371 Date: Aug. 11, 2022

Related U.S. Application Data
(60) Provisional application No. 62/975,084, filed on Feb. 11, 2020.
(60) Provisional application No. 63/148,045, filed on Feb. 10, 2021.

(51) Int. Cl. A61K 9/50; C12N 5/0786; A61K 35/15; A61K 35/28

(52) U.S. Cl. CPC A61K 9/5068; C12N 5/0645; A61K 35/15; A61K 35/28; C12N 2501/052; C12N 2501/24; C12N 2501/2304; C12N 2501/2314; C12N 2502/1157

(57) ABSTRACT

Compositions and methods described in this document make use of macrophage derived engineered vesicles (MEV) having specificity for delivery to a target environment, for use in modifying macrophage phenotype and/or treating a condition. Further disclosed are MEV derived from a specific phenotype encapsulating a therapeutic agent for targeted therapeutic delivery.

### RELATED APPLICATIONS

This application claims priority from U.S. Provisional Application Ser. Nos. 62/975,084 filed Feb. 11, 2020 and 63/148,045 filed Feb. 10, 2021, the entire disclosures of which are incorporated herein by this reference.

### GOVERNMENT INTEREST

This invention was made with government support under grant number DA038817 awarded by the National Institutes of Health. The government has certain rights in the invention.

### TECHNICAL FIELD

The presently-disclosed subject matter generally relates compositions and methods for modulating the phenotype of a macrophage in a targeted environment. In particular, certain embodiments of the presently-disclosed subject matter relate to a composition, and methods of making and using the composition, for modulating the phenotype of a macrophage in a targeted environment and/or to deliver cargo to the interior of a target cell to facilitate treatment of a condition.

### INTRODUCTION

The effective delivery of cargos such as fluorescent markers, genetic material, therapeutics, and proteins to the interior of the cell is important for the development of new therapeutics and for understanding biological function. Despite advances in areas such as gene delivery, targeted therapeutics, vesicle-based delivery systems, and the use of cell-penetrating peptides, the efficient transport of cargo across the cell membrane remains one of the primary challenges to the development of therapeutics.

Common strategies for accessing the interior of the cell utilize endocytic pathways. While this provides a relatively efficient means of crossing the cell membrane, it results in the trapping of cargo in endosomal vesicles. The vesicle must allow the cargo to be released to act upon the cell; however, such a feature results in a reduction in both the efficiency and the potential efficacy of the cargo. As such, ideal delivery vehicles would allow for the direct transport of cargo to the interior of the cell, bypassing endocytosis altogether.

Vesicles composed of phospholipid bilayers have shown promise as therapeutic delivery vectors capable of encapsulating the cargo and delivering it to the interior of target cells. Synthetic vesicles such as liposomes composed of phospholipid membranes are relatively easy to load and have shown promise as in vitro and in vivo intracellular delivery devices. However, applications are limited by a lack of biocompatibility, as liposomes are not capable of avoiding the immune system when being used for in vivo delivery.

Naturally occurring vesicles produced by cells are an attractive alternative. For example, Endogenous extracellular vesicles (EEVs) such as exosomes have received significant attention as therapeutic delivery vehicles to transport cargo across cell membranes because they are both nonimmunogenic and specifically target select cell types (e.g., they have the ability to specifically target the same cell type from which they originated.

While cell specificity addresses a major problem with targeted therapeutic delivery, the application of exosomes as cellular delivery devices is limited by low production efficiency and difficulty in loading with cargo. Despite these limitations, exosomes have been utilized for in vitro delivery of therapeutics and for gene delivery. Recently, vesicles generated from the membranes of organelles within the cells have been used as exosome-mimics and retained several of the targeting properties seen with exosomes. Nevertheless, the low yields and complex separation procedures still pose obstacles in the use of EEVs.

Macrophages are an essential component of the innate animal immune system. Macrophage function includes clearing waste materials such as cellular debris and participating in tissue repair and remodeling that occurs during wound healing. They also serve as a defense against bacterial infections and other pathogens largely through phagocytosis. Additionally, they are integral to the initiation of an adaptive immune response through their antigen presenting capabilities. As a result of this versatile role, macrophages exhibit a range of functional activities, which are often driven by stimuli in the surrounding environment.

Macrophages exist in a continuum of polarization states between a pro-inflammatory phenotype, classified as M1, and an anti-inflammatory phenotype, classified as M2. The polarization state is often mediated by environmental signals such as cytokines, fatty acids, and components from microorganisms such as lipopolysaccharides (LPSs). Pro-inflammatory macrophages are characterized by the production of nitric oxide and the release of high levels of inflammatory cytokines including IL-12, TNF-α, and IL-β.1 Anti-inflammatory macrophages secrete cytokines which can dampen the immune response such as IL-10 and IL-4.

The expression of specific macrophage cytokines is implicated in the progression of several disease states. For example, recent studies have shown that macrophages are involved in the progression of cancer, inflammatory diseases, and infectious diseases.

For example, in the tumor microenvironment, macrophages exhibit an anti-inflammatory phenotype and are known as alternatively activated or tumor-associated macrophages (TAMs). While IFN-γ and IL-12 release by pro-inflammatory macrophages have an anti-angiogenic effect and can block the formation of the new blood vessels in the tumor microenvironment, TAMs suppress production of these cytokines. Factors released by cancer cells in the tumor microenvironment cause TAMs to become tumor-supportive assisting in growth, tissue remodeling, angiogenesis, and metastasis. Tumor progression is further supported by TAMs, which produce reduced levels of the major histocompatibility complex (MHC)-II, which suppresses the anti-tumor adaptive immune response.

For another example, macrophages play a critical role in the inflammatory response such as during spinal cord injury (SCI). As the blood-brain barrier is compromised following SCI, peripheral macrophages rapidly invade the spinal cord and contribute to both pathological and reparative processes. While pro-inflammatory macrophages contribute to neurodegeneration and tissue loss after SCI, anti-inflammatory macrophages contribute to tissue remodeling and axon regeneration.

Control of macrophage phenotype through the ability to shift therapeutically between pro-inflammatory and anti-inflammatory polarizations has been proposed as a potential treatment for diseases such as some types of cancer and traumatic injury. Under different pathological conditions, macrophages exhibit heterogeneity across a continuum of polarization states. The ability to repolarize macrophages from one phenotype to another is a promising technique that might enable alternative forms of treatment for several diseases. For example, repolarizing TAMs toward a pro-inflammatory phenotype is an attractive means to sensitize cancer to immunotherapy. Similarly, repolarizing pro-inflammatory macrophages toward anti-inflammatory phenotypes, thereby reducing the potential neurotoxic effects of M1 macrophages, could be a promising approach for treating SCI and stroke.

Studies have shown that endogenous extracellular vesicles (EEVs) such as exosomes obtained from immune cells such as macrophages and dendritic cells possess the ability to repolarize TAMs to pro-inflammatory macrophages in the tumor microenvironment. Despite their promise in shifting macrophage phenotype as a therapeutic approach, EEV-based therapies are still challenged by low production yields and difficulties in separating target vesicles from other similarly sized vesicles.

Accordingly, there is a need in the art for improved tools for targeted delivery of cargo to target cells. For example, targeted delivery of chemotherapy to a cancer cell would be useful in treating solid tumors. Furthermore, there is a need in the art for tools to target macrophages in a specified environment to modulate polarization of the macrophages to a phenotype useful for treating a condition of interest. For example, repolarizing pro-inflammatory macrophages toward anti-inflammatory phenotypes, could reducing potential neurotoxic effects of the macrophages in the context of SCI and/or stroke. For another example, repolarizing anti-inflammatory macrophages toward pro-inflammatory phenotypes, could rendering cancer cells more sensitive to chemotherapy and/or immunotherapy.

### SUMMARY

The presently-disclosed subject matter meets some or all of the above-identified needs, as will become evident to those of ordinary skill in the art after a study of information provided in this document.

The presently-disclosed subject matter includes compositions and methods that specifically target cells and macrophages of interest. The compositions and methods of the presently-disclosed subject matter also allow for modulation of macrophage phenotype, which can be useful, for example, for repolarizing macrophages from pro-inflammatory (M1) to anti-inflammatory (M2), or vice versa, and treating various diseases such as traumatic injury and cancer.

The presently-disclosed subject matter includes also includes compositions and methods for effective delivery of cargo target cells and macrophages, and allow for cargo to be easily incorporated into the composition for delivery. The compositions and methods of the presently-disclosed subject matter avoid prior obstacles associated with low yields and complex separation procedures associated with endogenous extracellular vesicles.

The presently-disclosed subject matter generally relates compositions and methods for modulating the phenotype of a macrophage in a targeted environment. In particular, certain embodiments of the presently-disclosed subject matter relate to a composition, and methods of making and using the composition, for modulating the phenotype of a macrophage in a targeted environment and/or to deliver cargo to the interior of a target cell to facilitate treatment of a condition of interest.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 includes a schematic depiction of vesicle generation, loading, and isolation; cultured cells undergo nitrogen cavitation in the presence of cargo in free solution followed by serial centrifugation to generate purified vesicles. Vesicles serve as nanocarriers for hydrophilic cargo encapsulated during cavitation on the interior or for lipophilic cargo that can be embedded in the vesicle membrane.

FIGS. 2A-2C present results from cell-derived vesicle characterization; FIG. 2A is a wide-field fluorescence image of vesicles loaded with the fluorescent dye fluorescein; FIG. 2B includes a fluorescence correlation spectroscopy correlogram of vesicles used to determine vesicle concentration and relative yield; and FIG. 2C includes a plot of vesicle size distribution at different cavitation pressures as determined by dynamic light scattering.

FIGS. 3A-3E include results showing cell-targeting specificity; FIG. 3A includes a comparison of HEK vesicles delivered to HEK cells (black) versus HEK vesicles delivered to A549 cells (grey); FIG. 3B includes a comparison of RAW vesicles delivered to RAW cells (black) versus RAW vesicles delivered to A549 cells (grey); FIG. 3C includes a comparison of HCT vesicles delivered to HCT cells (black) versus RAW vesicles delivered to HCT cells (grey); FIG. 3D includes a wide-field fluorescence image of DiI-labeled RAW vesicles delivered to RAW cells after 2.5 h showing clear loading; and FIG. 3E includes a wide-field fluorescence image of DiI-labeled RAW vesicles delivered to A549 cells after 2.5 h showing limited cellular uptake. Norm ID is the integrated density of the image normalized to the time 0 value. Each data point is the average of five experiments. A Student's t-test was used to determine significance between end points. Each end point was significant with a p value of <0.001.

FIG. 4 illustrates the efficacy of cisplatin-loaded vesicles, and includes a comparison of cell growth at time 0, 24, 48, and 72 h for A549 cells with no treatment (black; first bar in each time category), treated with empty vesicles (light gray; second bar in each time category), with cisplatin-loaded vesicles (dart gray; second bar in each time category), and free cisplatin in solution (medium gray; fourth bar in each time category). Empty vesicles have no effect on cell growth while both free cisplatin and loaded vesicles show similar efficacy in killing A549 cells. Each data point is the average of five experiments. A Student's t-test was used to determine significance. The asterisk “*” indicates a p value of <0.001.

FIGS. 5A-5D illustrate the results of vesicle-based gene delivery; FIG. 5A includes a wide-field image of HEK cells in the absence of plasmid; FIG. 5B includes a wide-field image of HEK cells after exposure to Dendra2 plasmid-loaded vesicles showing clear cellular delivery based on the expression of the fluorescent protein; FIG. 5C includes a Brightfield image of HEK cells in the absence of plasmid; and FIG. 5D includes a Brightfield image of HEK cells after exposure to plasmid loaded vesicles.

FIG. 6 presents results of confocal imaging to illustrate vesicle delivery, and includes a confocal image of HEK 293 cells after the delivery of vesicles loaded with fluorescein (interior) and DiD (cell membrane). The interior of the cell is filled with fluorescein and vesicles can be seen on the cell surface.

FIGS. 7A-7D depict mice bearing A549 xenografts on the right shoulder (dashed ovals), which were injected with dye (DiR) alone (FIG. 7A), dye-labeled vesicles derived from HEK cells (FIG. 7B), dye-labeled vesicles derived from A549 cells (FIG. 7C), and dye-labeled vesicles derived from RAW264.7 cells (FIG. 7D), demonstrating RAW vesicles specifically targeted the A549 xenograft.

FIG. 8A is a schematic illustrating an approach of generating macrophage-derived engineered vesicles (MEV) from Polarized Bone Marrow-Derived Macrophages. As depicted, fully differentiated unstimulated macrophages (M0) are polarized into either pro-inflammatory macrophages (M1) or anti-inflammatory macrophages (M2). Nitrogen cavitation is then used to fragment the cellular membranes of these cells generating M1-engineered vesicles (M1EVs) or M2-engineered vesicles (M2EVs). Vesicles are then separated from cellular fragments by serial centrifugation. These vesicles are then delivered to either unstimulated or polarized macrophages to shift the polarization toward the polarization type of the MEVs.

FIG. 8B includes a schematic illustrating an exemplary approach of using MEVs from macrophages polarized to a M1 pro-inflammatory phenotype, which are delivered to an animal with tumor xenograft, resulting in the predominate anti-inflammatory macrophages (M2) present in the tumor microenvironment being shifted to a pro-inflammatory phenotype (M1).

FIG. 8C includes a schematic illustrating an exemplary approach of using MEVs for personalized cancer treatment.

FIGS. 9A-9E include results from macrophage-derived engineered vesicles (MEV) characterization; FIG. 9A includes a fluorescence image of MEVs loaded with a fluorescent dye (fluorescein) during vesicle generation illustrating the principle of encapsulation of cargo by MEVs; FIG. 9B includes a fluorescence image of MEVs labeled with the lipophilic dye, DiI; FIG. 9C includes a size distribution of pro-inflammatory MEVs (M1EVs) measured by nanoparticle tracking analysis, and FIG. 9D includes a size distribution of anti-inflammatory MEVs (M2EVs), and the effective diameter of the vesicles generated by nitrogen cavitation was between 100-200 nm; and FIG. 9E includes a bar graph showing the effective diameter of M1 vesicles in PBS for 3 days measured using dynamic light scattering.

FIGS. 10A-10C illustrate the efficacy of M1EVs as systems to deliver cargo to the interior of cells; FIG. 10A includes a confocal image showing M0 macrophages exposed to fluorescein (soluble dye)-loaded vesicles exhibit fluorescence after 2 hours of interaction with the fluorescein-loaded vesicles; FIG. 10B includes a confocal image of DiI-labelled MlEVs delivered to M0 macrophages after 2 hours of interaction showing clear uptake of vesicles by macrophages; and FIG. 10C includes a 3D confocal image of an M2 macrophage after delivery of fluorescein (interior)-loaded M1EVs labeled with DiI (lipid bilayer), showing clear uptake of vesicles on the surface and inside by macrophages.

FIGS. 11A-11G illustrate macrophage targeting specificity; FIGS. 11A-11D include widefield fluorescence images of M2 macrophages showing the time-dependent uptake of DiI-labeled M1EVs by M2 macrophages, scale bar=30 μm; FIG. 11E includes a comparison of M1EVs delivered to M1 macrophages (black) vs M1EVs delivered to M2 macrophages (gray); FIG. 11F includes a comparison of M2EVs delivered to M1 macrophages (black) vs M2EVs delivered to M2 macrophages (gray); FIG. 11G includes a comparison of M2 Macrophages with M1EV delivery (Free), M2 macrophages incubated with dynasore (80 μM) for 30 min prior to M1EV addition (Dyna), and M2 macrophages with M1EV delivery in the presence of DMSO (delivery vehicle) (DMSO). Each data point is the average of five independent replicates (n=5). Norm. ID is the mean integrated density of the image normalized to the mean integrated density value of M2 macrophages before adding vesicles. The data are presented as the mean±SEM. **p<0.01 indicates a significant difference in the vesicle uptake by macrophages at respective time points.

FIGS. 12A and 12B illustrate reprogramming macrophage polarization with MEVs; FIG. 12A includes measurement of the pro-inflammatory cytokines and nitric oxide (NO) released by M0, M1, and M2 macrophages compared to the production of cytokines released after M1EV delivery to M2 and M0 macrophages; both M0 and M2 macrophages are polarized toward an M1 phenotype upon interaction with M1EVs in vitro; and FIG. 12B includes measurement of pro-inflammatory cytokines and NO expression by M0 and M1 macrophages when incubated with M2EVs for 24 h in vitro; pro-inflammatory cytokines released by M1 macrophages are significantly reduced upon interaction with M2EVs, which shows that M2EVs are capable of reprogramming M1 macrophages toward an M2 phenotype. Each data point is the average of at least three experiments (n=3). The data are presented as the mean±SEM.

FIG. 13 includes quantification of pro-inflammatory cytokines present on M1EVs. M1EVs were freeze-thawed (M1EVs F.T) to break them and release the encapsulated cargo if present inside vesicles.

FIGS. 14A-14G include quantification of cytokine released by M1 microglia, M2 microglia and M2 microglia that were incubated with M1EVs. Each data point is the average of at least 3 experiments. The data are presented as the mean±SEM. One-Way ANOVA was done to test the statistical significance of the results.

FIGS. 15A and 15B illustrate macrophage-mediated neurotoxicity; FIG. 15A includes the effect of macrophage-conditioned media on the viability of differentiated N2a cells was determined using a cell viability assay for control cells with growth media (control), for the supernatant from M1 macrophage culture (M1), and for the supernatant from M1 macrophage culture after treatment with M2EVs (M2EVs on M1); FIG. 15B includes a comparative study of the ability of M2EVs and AZM in solution (10 μM) to reprogram M1 macrophages toward an M2 phenotype. Each data point is the average of five independent replicates (n=5). **p<0.01 indicates that the results are statistically significant. The data are presented as the mean±SEM.

FIG. 16 illustrates specificity of targeting of MEVs on macrophages and A549 cells. Each datapoint is the average of five independent replicates. Norm. ID is the mean integrated density of the image normalized to the mean integrated density value of macrophages prior to vesicles addition. The data are presented as the mean±SEM (n=5). **P<0.01 indicates a significant difference in the vesicle uptake by macrophages and A549 cells at respective time points.

FIGS. 17A-17C illustrate the efficacy of MEVs as biological nanocarriers; FIG. 17A includes a comparison of targeting specificity of cisplatin-loaded M0, M1, and M2-engineered vesicles to A549 cells. Each data point is the average of five independent replicates (n=5). ANOVA with post hoc Tukey's HSD was used to test the significance of the results. **p<0.01 indicates that the results are statistically significant. The data are presented as the mean±SEM. FIG. 17B compares the effect of free cisplatin or cisplatin loaded MEVs on viability of adenocarcinomic human alveolar basal epithelial cells (A549). FIG. 17C includes results from mice bearing A549 xenografts that were injected with DiR labelled M1EVs, demonstrating that M1EVs can reach the tumor of the mice.

FIGS. 18A-18C include data establishing that M1 MEVs repolarize M2 macrophages and have anticancer activity. FIG. 18A includes quantification of the cytokine TNFα released by M1 macrophages, M2 macrophages, and M2 macrophages that were incubated with M1EVs. FIGS. 18B and 18C present viability of Caov-3 cells alone (FIG. 18B) or M2 macrophages plus Caov-3 cells (FIG. 18C), which were treated with 20% or 10% M1 MEVs for 24 hrs.

FIGS. 19A and 19B illustrate that M1 MEVs from multiple sources are able to re-polarize human M2 macrophages. FIG. 19A M1 MEVs derived from RAW cells, mouse bone marrow-derived cells, and human PBMC-derived macrophages are able to re-polarize M2 macrophages. FIG. 19B Human macrophages repolarized using human M1 MEVs show significant shifts in TNFα secretion.

FIG. 20 is a schematic illustration showing exemplary markers associated with assessing M1 MEV surface proteins repolarizing M2 macrophages.

FIG. 21 includes results of a Western blot of M1 macrophages (lane 1), isolated M1EVs (lane 2), and M2 macrophages (lane 3).

FIG. 22 includes results of a Western blot of M1 macrophages (lane 1), isolated M1EVs (lane 2), and M2 macrophages (lane 3).

FIG. 23 includes results of a Western blot of M1 macrophages (lane 1), isolated M1EVs (lane 2), and M2 macrophages (lane 3).

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes compositions and methods using macrophage-derived engineered vesicles (MEV) having specificity for delivery to a target environment, for use in modifying macrophage phenotype and/or treating a condition.

Some embodiments of the presently-disclosed subject matter include a method of making a macrophage-derived engineered vesicle (MEV). In some embodiments, the method includes providing a first macrophage of a first phenotype, fragmenting a cell membrane of the first macrophage, allowing the fragmented membrane to assemble into a first phenotype MEV derived from the first macrophage. In some embodiments, the method can include incubating the first phenotype MEV derived from the first macrophage with a second macrophage of a second phenotype, thereby shifting the phenotype of the second macrophage to the first phenotype.

Some embodiments of the presently-disclosed subject matter include a method of modifying a phenotype of a macrophage, which involves providing a macrophage of a second phenotype, and incubating the macrophage with a macrophage-derived engineered vesicle (MEV) of a first phenotype, thereby shifting the macrophage to a first phenotype.

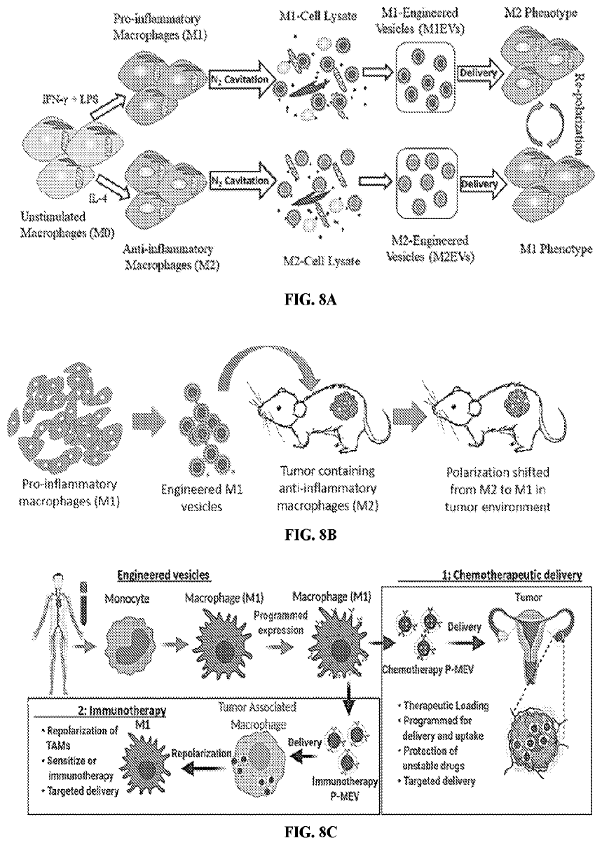

In this regard, with reference to FIGS. 8A and 8B, if the first phenotype is M1, the first phenotype MEV would be M1EVs. If the second phenotype is M2, then when the M1EVs are delivered to the M2 macrophage, the macrophage will repolarize to the M1 phenotype. Conversely, with reference to FIG. 8A, if the first phenotype is M2, the first phenotype MEV would be M2EVs. If the second phenotype is M1, then when the M2EVs are delivered to the M1 macrophage, the macrophage will repolarize to the M2 phenotype.

Some embodiments of the presently-disclosed subject matter involve making an MEV from a macrophage. As described herein, to make the MEV, the cell membrane of the macrophage is fragmented. The fragmentation can be achieved using nitrogen cavitation, for example, as disclosed herein. The fragmented membrane is placed in an assembly solution, where it assembles into the MEV. The assembly solution can optionally contain cargo, such that MEV encapsulates the cargo during assembly. In such embodiments, the cargo can be selected based on the desired application. For example, cargo could include genetic material, therapeutics, protein, and fluorescent markers. In some embodiments, the MEV has a therapeutic application, and thus, it can be desirable to select a therapeutic agent. For example, if the therapeutic application is to cancer, a chemotherapeutic agent could be selected such as, for example, cisplatin or a checkpoint inhibitor such as pembrolizumab. For another example, in the case of an inflammatory disease, it could be useful to select an anti-inflammatory agent.

With further reference to embodiments of methods that involve making an MEV from a macrophage, the macrophage can be obtained from a target environment or source. The source of the macrophage will depend on the desired application. In some embodiments, the target environment could be an in vitro environment, and in other embodiments the target environment could be an in vivo environment.

For example, in some embodiments the macrophage is obtained from human peripheral blood mononuclear cell-derived monocytes. For another example, in some embodiments, the macrophage is obtained from bone marrow. In this regard, where the obtained macrophage is unstimulated (designated M0), the macrophage can be stimulated to an M1 macrophage with macrophages with lipopolysaccharide (LPS) and interferon gamma (IFN-γ), or it can be stimulated to an M2 macrophage with interleukin 4 (IL-4) and/or interleukin-13 (IL-13).

In some embodiments, the macrophage is obtained from an in vivo environment. The skilled artisan will recognize, upon study of this document, that source of the macrophage being a particular in vivo target environment can be particularly beneficial for enhancing targeting specificity.

In some embodiments, the method further involves contacting the MEV with the target environment or environment to which targeted delivery of the MEV is desired. In such embodiments in which the target environment is in vivo environment, the contacting could involve administering the MEV to a subject. The particular in vivo environment could be the site of a condition in a subject. The condition could be a cancer, such as ovarian, lung, colorectal, a condition of the central nervous system, a wound, an inflammatory disease, an infectious disease, a traumatic injury such as a spinal cord injury, or an ischemic event such as a stroke. In this regard, and the site could be associated with the relevant condition. For example, if the condition is a cancer, then the in vivo environment could be the cancer or tumor micro environment.

FIG. 8B depicts administration of M1EVs to a mouse having a tumor. In this regard, the tumor micro-environment containing anti-inflammatory macrophages (M2) is the target environment to which delivery of the M1EVs is desired. As illustrated, upon delivery, the M1EVs repolarize the M2 macrophages to M1, facilitating treatment of the cancer.

With reference to FIG. 8C, the presently-disclosed subject matter has particular utility in the context of personalized medicine. A human subject who is an ovarian patient is depicted as the source of the macrophages. In the depicted example, the macrophages are obtained from human peripheral blood mononuclear cell-derived monocytes from the patient, which are used to prepare M1EVs. In this depicted example, at least some of the M1EVs are encapsulating cargo (See panel labeled “1: chemotherapeutic delivery”). These chemotherapy-loaded MEVs are able to specifically target the ovarian tumor microenvironment to deliver the chemotherapy direct into the tumor cells. Meanwhile, the M1EVs (either unloaded or loaded with cargo) have a immunotherapeutic approach (See panel labeled “2: Immunotherapy”). The M1EVs are specifically delivered to the tumor micro-environment, where they facilitate repolarization of M2 TAMs to M1 macrophages, sensitizing the cancer to immunotherapy.

The presently-disclosed subject matter further includes a composition that comprises a macrophage-derived engineered vesicle (MEV) having a first phenotype, derived from a target environment, and optionally encapsulating cargo, as disclosed herein.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9): 1726-1732).

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “subject” refers to a target of administration. The subject of the herein disclosed methods can be a mammal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A “patient” refers to a subject afflicted with a disease or disorder. The term “patient” includes human and veterinary subjects.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term MEV refers to macrophage derived engineered vesicles. MEVs may be of the M1 or M2 phenotype or predominately of the M1 or M2 phenotype. M0 bone marrow derived macrophages may be used to generate either M1 or M2 macrophages.

As used herein chemotherapeutic refers to therapeutics used to treat cancer. Specific non-limiting examples include Altretamine, Bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa, Trabectedin, Carmustine, Lomustine, Streptozocin, Azacitidine, 5-fluorouracil (5-FU), 6-mercaptopurine (6-MP), Capecitabine (Xeloda), Cladribine, Clofarabine, Cytarabine (Ara-C), Decitabine, Floxuridine, Fludarabine, Gemcitabine (Gemzar), Hydroxyurea, Methotrexate, Nelarabine, Pemetrexed (Alimta), Pentostatin, Pralatrexate, Thioguanine, Trifluridien/tipiracil combination, Daunorubicin, Doxorubicin (Adriamycin), Doxorubicin liposomal, Epirubicin, Idarubicin, Valrubicin, Bleomycin, Dactinomycin, Mitomycin-C, Mitoxantrone, Irinotecan, Irinotecan liposomal, Topotecan, Etoposide (VP-16), Mitoxantrone (also acts as an anti-tumor antibiotic), Teniposide, All-trans-retinoic acid, Arsenic trioxide, Asparaginase, Eribulin, Hydroxyurea, Ixabepilone, Mitotane, Omacetaxine, Pegasparaginase, Procarbazine, Romidepsin, and Vorinostat.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: Cisplatin Delivery

A549 cells (60 million) were used to generate vesicles for delivery. Cells were scraped from culture in 20 mL of sucrose buffer containing protease inhibitor. The cell solution was collected in a 50 mL conical tube and pelleted at 2000 rpm at 25° C. for 2 min.

The solution was aspirated off, and the pellet was resuspended in 8 mL of 8.33 mM cisplatin in sucrose buffer solution or 20 mg cisplatin/8 mL sucrose buffer solution plus protease inhibitor.

The cell solution was fragmented using N2 cavitation at 300 psi at 4° C. for 5 min. The resulting cell slurry was centrifuged at 4000 g for 10 min at 4° C. The supernatant from centrifugation was transferred to a 25 mL ultracentrifuge tube for 10,000 g centrifugation for 20 min at 4° C. The supernatant from the UCF tube was then transferred to a new 25 mL ultracentrifuge tube and centrifuged at 100,000 g for 60 min at 4° C. The pellet in the UCF tube was washed with 500 μL of sucrose solution, and the residual solution was pipetted out and discarded. Seven hundred fifty microliters of sucrose buffer solution was added to the UCF tube, and the pellet was resuspended via pipetting. Empty vesicles were generated in the exact same way except in the absence of cisplatin.

A549 cells (30,000) were plated in each well of a 96-well plate and allowed to seed for 24 h. During that time, the growth media was exchanged for 250 μL of fresh HEK media containing either cisplatin in solution (4.17 mM), cisplatin-loaded vesicles, empty vesicles, or untreated media cisplatin solution in HEK media. The media was aspirated off. Two hundred microliters of Optimem was added followed by 20 μL of alamar blue. The plate was left to incubate at 37° C. with gentle tapping every 10 min for 40-45 min to ensure uniform turnover to a brilliant purple. The plate was read using a FlexStation plate reader.

### Example 2: Gene Delivery

HEK cells (32 million) were scraped with 10 mL of sucrose buffer solution with protease inhibitor. All of the cell solution was collected into a 15 mL conical tube and pelleted at 2000 rpm at 25° C. for 2 min. The solution was aspirated off and resuspended in 3 mL sucrose buffer solution plus protease inhibitor. Plasmid (75 pg of Dendra2) was added to the N2 cavitation chamber. The solution was fragmented using N2 cavitation at 600 psi at 4° C. for 5 min. The cell slurry was centrifuged at 4000 g for 10 min at 4° C. The supernatant from centrifugation was transferred to a 25 mL ultracentrifuge tube and centrifuged at 10,000 g for 20 min at 4° C. The supernatant from the UCF tube was then transferred to a new 25 mL ultracentrifuge tube and centrifuged at 100,000 g for 60 min at 4° C. The pellet in the UCF tube was washed with 500 μL sucrose solution, and the residual solution was pipetted out and discarded. One milliliter of sucrose buffer solution was added to the UCF tube, and the pellet was resuspended via pipetting. Two hundred fifty microliters of this solution was added to 30,000 HEK cells plated on glass bottom dishes. After 24 h, the cells were rinsed and then imaged at 48 h using a wide-field microscope with a 488 nm band-pass filter for excitation.

### Example 3: Vesicle Delivery

Vesicles were prepared as described above. The vesicle solution was mixed in an Eppendorf tube (1 mL) with 2 μL of 2 mM DiI and left to incubate for 30 min at 37° C. Labeled vesicles were separated from free fluorescein/DiI using a PD MidiTrap equilibrated with sucrose buffer solution. One hundred eighty microliters of the vesicle solution was added to cells plated on glass bottom dishes. Cells were imaged using an excitation wavelength of 561 nm after 2 h of incubation at 37° C. Control studies to determine the leaching of encapsulated and membrane-bound fluorophores were performed. Vesicles with either DiI or fluorescein were incubated in solution for 4 h to mimic the conditions of cell labeling. After 4 h, the vesicles were pelleted using ultracentrifugation. The supernatant was then added to the cell culture to determine the presence of any free dye. No visible fluorescence was observed for control studies with fluorescein or with DiI.

### Example 4: In Vivo Xenograft

Athymic nude mice were injected with A549 cells (NSCLC, immortalized) in the right shoulder and monitored for 3-4 weeks until measurable xenograft tumors were observed. RAW cells were cultured in vitro by standard methods. Cell-derived vesicles were manufactured by the Richards lab as described above and loaded with DiR near-infrared fluorescent dye. Prepared vesicles were administered, and mice were then imaged approximately 24, 48, and 72 hours later. Mice were anesthetized for imaging using isoflurane. Epifluorescence was measured using an IVIS Spectrum In vivo Imaging System (PerkinElmer). Vesicles were labeled with DiR near-infrared fluorescent dye, which was excited at 710 nm, with emission measured at 780 nm. Fluorescent signal intensity (i.e., total radiant efficiency) within regions of interest (ROI) were quantified using Living Image software (PerkinElmer), correcting for background fluorescence using distal site ROI within the same mouse.

RAW264.7 cells (100 million) were scraped in 40 mL of sucrose buffer solution with protease inhibitor. Vesicles were prepared as described above. The cells were labeled using DiR at 2 μM for 30 min at 37° C. Samples were injected intravenously to the mouse, through the tail vein. Approximately 1×10¹¹ vesicles were delivered per injection. All animal experiments were repeated three times.

### Example 5: Determining Cisplatin Concentration

Vesicles were generated as described above in the presence of 8.33 mM cisplatin in sucrose buffer solution. To release cisplatin from the vesicles, they were treated with 5 μL (1%) Triton X-100 followed by 500 μL of 70% nitric acid and incubated on a heat block at 6° C. for 2 h. The solution was diluted to 5 mL of 1% nitric acid and analyzed using ICP-OES (Varian Vista Pro).

To determine the amount of leakage into the solution from the vesicles, the supernatant of the vesicle solution was collected after the vesicles were pelleted using ultracentrifugation. The supernatant was then diluted in 5 mL of 1% nitric acid and analyzed using ICP-OES. Separately, vesicles were stored for 1, 2, and 3 days. At each time point, the vesicles were pelleted, and the supernatant was collected and analyzed for cisplatin using ICP-OES. A standard curve was generated using standard concentrations of platinum in 1% nitric acid ranging from 1 ppm to 10 ppb. Ytterbium was used as in the internal standard to compensate for the internal drift of the instrument.

### Example 6: Dynamic Light Scattering

Vesicles were prepared as described above. The vesicle solution was then diluted (1:20) and analyzed using DLS.

### Example 7: Cell Specificity

HEK vesicles onto HEK and A549 cells: 64 million HEK were used to generate vesicles for delivery. Cells were scraped from culture in 20 mL of sucrose buffer containing protease inhibitor. The cell solution was collected in a 50 mL conical tube and pelleted at 2000 rpm at 25° C. for 2 min. The solution was aspirated off such that the final volume was 10 mL. The cell solution was fragmented using N2 cavitation at 300 psi at 4° C. for 5 min. The resulting cell slurry was centrifuged at 4000 g for 10 min at 4° C. The supernatant from centrifugation was transferred to a 25 mL ultracentrifuge tube for 10,000 g centrifugation for 20 min at 4° C. The supernatant from the UCF tube was then transferred to a new 25 mL ultracentrifuge tube and centrifuged at 100,000 g for 60 min at 4° C. The pellet in the UCF tube was washed with 500 μL of sucrose solution, and the residual solution was pipetted out and discarded. One thousand microliters of sucrose buffer solution was added to the UCF tube, and the pellet was resuspended via pipetting. Two microliters of 1 mM DiI was added to the resuspension and left to incubate for 30 min at 37° C. After this time, the vesicles were purified from the free dye using a PD MidiTrap. Fifty microliters of the purified vesicles was added into each glass bottom dish containing 90,000 HEK or A549 cells plated 24 h prior.

This procedure was repeated for RAW vesicles on RAW and A549 cells using 54 million cells to generate vesicles.

The above protocol was repeated for HCT vesicles on HCT cells and RAW vesicles on HCT cells using 70.4 and 56.8 million cells, respectively, to generate vesicles. Since HCT cells grow at a faster rate than most other cell types that were used, 50,000 cells were plated onto glass bottom dishes instead of the traditional 90,000 cells.

### Example 8: Fluorescence Correlation Spectroscopy

Forty million A549 cells were scraped from the culture in 20 mL of sucrose buffer containing protease inhibitor. The cell solution was collected in a 50 mL conical tube and pelleted at 2000 rpm at 25° C. for 2 min. The solution was aspirated off such that the final volume was 10 mL. The cell solution was fragmented using N2 cavitation at 250 psi at 4° C. for 5 min. The resulting cell slurry was centrifuged at 4000 g for 10 min at 4° C. The supernatant from centrifugation was transferred to a 25 mL ultracentrifuge tube for 10,000 g centrifugation for 20 min at 4° C. The supernatant from the UCF tube was then transferred to a new 25 mL ultracentrifuge tube and centrifuged at 100,000 g for 60 min at 4° C. The pellet in the UCF tube was washed with 1 mL of PBS, and the residual solution was pipetted out and discarded. Three hundred microliters of PBS was added to the UCF tube, and the pellet was resuspended via pipetting. DiI (2 μM) was added to the resuspension and left to incubate for 40 min at 37° C. After this time, the vesicles were purified from the free dye using a 450 nm Corning sterile syringe filter only after the filter was pre-saturated with 150 μL of PBS. The solution was then diluted 1:2, and then 20 μL was placed onto a coverslip mounted on a 60× water objective. A 532 nm laser (45 mW) was used as an excitation source. A picoquant PicoHarp 300 photon counting system was used to time tag photon arrival events.

### Example 9: Animals for Bone Marrow Cell Extraction

Two to five-month-old wild-type C57BL/6 mice were used to extract bone marrow cells. Animals were properly accommodated in IVC cages by providing enough food and water. All experiments were performed following the guidelines of the National Institute of Health and were approved by the Institutional Animal Care and Use Committee at the University of Kentucky.

### Example 10: Cell Culture

BMDMs were isolated from both tibias and femurs of wild-type mice at 2-5 months of age as previously reported. Briefly, mice were first anesthetized and then killed by cervical dislocation. After removing femurs and tibias from the carcass, the bone marrow was extracted using a 10 mL syringe loaded with Roswell Park i Institute (RPMI) Medium into a 50 mL centrifuge tube. The bone marrow in media was then triturated with an 18-gauge needle until a single cell suspension was obtained, followed by centrifugation at 1,200×g for 5 min. The supernatant was carefully removed, and cells were resuspended in 4 mL of RBC lysis buffer (0.15 M NH4C1, 10 mM KHCO3, and 0.1 mM Na4EDTA), followed by swirling by hand for 3 min. 6 mL of RPMI media was then added, followed by centrifugation at 1200×g for 5 min. The supernatant was aspirated off, and the cells were resuspended in differentiation media (RPMI supplemented with 10% fetal bovine serum (FBS), 1% penicillin/streptomycin (PS), 1% (4-(2-hydroxyethyl)-1-piperazineethanesulfonic acid), 0.001% β-mercaptoethanol, 1% glutamine, and 20% supernatant from sL929 cells) and plated in T-175 cell culture flasks in differentiation media. sL929 cell lines were maintained in RPMI media supplemented with 10% FBS, 1% PS, and 1% glutamine. The supernatant from sL929 cells contains the macrophage-colony stimulating factor (MCSF), which is essential for differentiating bone marrow cells into macrophages. Differentiation media were replaced on days 2, 4, and 6, and the cells were replated on day 7 at a cell density of 1×10⁶ cells/mL in replating media [Dulbecco's modified Eagle's medium (DMEM) supplemented with 10% FBS, 1% glutamine, and 1% PS). On day 8, cells were stimulated to M1 [LPS (20 ng/mL; Invivogen)+IFN-γ (20 ng/mL; eBioscience)] or M2 [IL-4 (20 ng/mL); eBioscience] macrophages, while the unstimulated macrophages from day 7 were termed M0 macrophages. For cytokine analysis, the supernatant from stimulated cells, MCM, was collected after 24 h. Vesicles were added after 12 h of stimulation, and the supernatant was collected after 24 h of vesicle addition to M1 or M2 macrophages. The MCM obtained were collected into Eppendorf tubes and stored at −80° C. until the analysis was done.

Primary cultures of microglia were prepared from postnatal P2 to P4 pups from C57BL/6 mice. Briefly, pups were decapitated, and brains were kept in Petri dishes filled with ice-cold Hank's Balanced Salt Solution (Ca2+, Mg2+, NaHCO3, and phenol red). Brains were dissected, and the hippocampal region was extracted for microglia isolation and culture. The tissues were then minced, and the cell suspension was made. The cell suspension was treated with 2.5% trypsin (quality biological), incubated, and finally resuspended in the astrocyte culture media containing DMEM with 10% FBS and 1% PS. Cells were incubated at a density of 2 million on a poly-1-lysine-coated T75 flask containing astrocyte culture media. Cell culture media were changed every 3 days until the flask was confluent with cells. Microglia were detached from astrocytes and oligodendrocytes by shaking the flasks for 30 min at a speed of 180 rpm.

The mouse neuroblastoma cell line (also known as Neuro-2a or N2a) was maintained in the N2a cell culture medium composed of 44% DMEM, 45% OPTI-MEM reduced-serum medium, 10% FBS, and 1% PS. A total of 40,000 N2a cells were plated in each well of a 96-well plate in N2a media supplemented with 20 μM retinoic acid (Sigma-Aldrich) and allowed to differentiate for 24 h. Retinoic acid helped N2a cells to differentiate into cells with neuron-like properties. On day 1, the differentiation media were exchanged for 100 μL of various MCM in 20 μM retinoic acid and two controls with and without 20 μM retinoic acid. Cells were further incubated for 48 h, and the neurotoxicity of MCM was evaluated using an alamar blue cell proliferation assay.

Human lung cancer (A549) cells were maintained in the A549 cell culture medium composed of 89% DMEM, 10% FBS, and 1% PS. A total of 40,000 A549 cells were plated in each well of a 96-well plate and left to incubate for 12 h at 37° C. After 12 h, the old growth media were removed carefully, being sure not to disturb the cells, and were exchanged with A549 cell media containing cisplatin-loaded macrophage (M0, M1, and M2)-engineered vesicles or empty (M0, M1, and M2) vesicles. After 24 h of incubation at 37° C., the media were aspirated off and 100 μL of Optimem was added, followed by 20 μL of alamar blue for the cell viability assay.

### Example 11: Cell Viability Assay

For cytotoxicity assays, the cell media from each well of a 96-well plate were exchanged for 100 μL of Optimem (Invitrogen), followed by the addition of 20 μL of alamar blue. Cells were then incubated for 35-45 min until a uniform purple coloration was developed. The resulting fluorescence was measured using a Tecan 96-well plate reader equipped with an excitation filer set to 535 nm and the emission filter set to 595 nm. All measurements were done in quintuplicate (five different wells), and at least three independent experiments were carried out.

### Example 12: MEV Isolation

MEV formation involves disrupting cellular membranes into nanoscale fragments. Nitrogen decompression involves maintaining cells in a pressurized chamber (300 psi). The rapid release of pressure ruptures and segments cellular membranes, which spontaneously form vesicles in aqueous solutions. Cell-derived vesicles maintain the same surface proteins found in the membrane of the parent cell. Because vesicles are generated in the presence of the existing extracellular solution, they can encapsulate anything in that solution at the time of formation.

Completely differentiated macrophages from day 8 were used to generate MEVs. The macrophage cell media were aspirated off from the flask containing macrophages, and the cells were first washed with PBS. 3 mL of PBS was further added to each flask, and cells were detached by scraping them, followed by resuspension in PBS. The cell suspension from all flasks was first collected into a 50 mL tube, and the total number of cells was counted using a hematocytometer.

The cell slurry collected in the previous step was then centrifuged at 1200 rpm at 4° C. for 5 min, and the obtained pellets were resuspended in 10 mL of PBS supplemented with the protease inhibitor. To fragment the cellular membrane and generate the vesicles, cells were then subjected to a pressure of 300 psi for 5 min in a prechilled nitrogen gas decompressor (Parr Instruments Company, IL, USA) on ice. The pressure was rapidly released to generate fragmentation resulting in vesicles. The fragmented cell mixture including vesicles was centrifuged at 4000×g for 10 min at 4° C. The pellet obtained was discarded, but the supernatant was centrifuged at 10,000×g for 20 min at 4° C. The supernatant was again subjected to ultracentrifugation at 100,000×g for 60 min at 4° C. to pellet the remaining nanovesicles. The pellet was washed five times with PBS before being resuspended in 500 μL of the PBS buffer.

### Example 14: MEV Characterization

MEVs were generated by nitrogen cavitation, followed by a series of centrifugation steps as discussed above. The mean diameter, concentration, and zeta potential of MEVs were determined via nanoparticle tracking analysis (NTA) using a Nanosight 300 and a ZetaView PMX-120. Similarly, MEV stability was determined using dynamic light scattering (DLS). A ZetaPALS potential Analyzer (Brookhaven Instruments) was used to obtain the DLS measurements.

### Example 15: MEV Labeling

Cells were detached from the flask and counted and resuspended in 9.9 mL of PBS. 100 μL of 100 mM fluorescein was added to the cell suspension so that the final concentration of fluorescein becomes 1 mM in the cell suspension. The cell solution was fragmented using nitrogen cavitation, and the vesicle pellet was obtained. The pellet was then washed with PBS to remove any unincorporated fluorescein inside the vesicle. Vesicles were then resuspended in 1 mL of PBS and transferred to a clean ultracentrifuge (UCF) tube where the vesicle suspension was diluted to 4 mL in PBS. For the complete removal of the free dye, the diluted vesicle suspension was recentrifuged at 100,000×g for 60 min at 4° C. The supernatant from centrifugation was discarded, and the pellet was washed with 1 mL of the PBS buffer. 500 μL of PBS was added to the UCF tube, and the pellet was resuspended by pipetting several times. DiI was then added to the vesicle resuspension such that the final concentration of the dye becomes 2 μM and left to incubate for 30 min at 37° C. DiI is a lipophilic dye which gets incorporated into the lipid bilayer of the vesicle. The free dye molecules were separated from the fluorescently labeled vesicles using a size exclusion spin column (PD MidiTrap column). The column was equilibrated first by running 15 mL of PBS through the column and the column was centrifuged at 1000 g for 2 min to remove any remaining PBS from the column. Then, 500 μL of the vesicle solution was added carefully onto the center of the column from the top and centrifuged at 1000 g for 2 min to obtain DiI-labeled vesicles loaded with fluorescein.

### Example 16: MEV Imaging

DiI or fluorescein-labeled vesicles were generated as discussed previously and deposited onto a glass bottom dish before imaging them using fluorescence microscopy. DiI-labeled vesicles were imaged using a 532 nm laser of a 1.9 mW power with a gain of 990 and an exposure time of 200 ms. Similarly, fluorescein-loaded vesicles were imaged using a 488 nm laser of a 0.8 mW power with a gain of 990 and an exposure time of 200 ms.

### Example 17: Confocal Imaging

A Nikon MR laser scanning confocal microscope equipped with a 60× oil objective was used for confocal imaging of macrophages that had taken up dye-labeled vesicles. Thus, the obtained images were analyzed with Nikon image processing software.

### Example 18: MEV Uptake

100 million M1 and 110 million M2 macrophages were used to prepare M1EVs and M2EVs, respectively, for the study of MEV uptake by M1 or M2 macrophages. MEVs were generated and labeled with DiI as mentioned previously. From total 500 μL of each vesicle suspension, 50 μL of DiI-labeled vesicles was then added separately to each glass bottom dish containing 90,000 M1 or M2 macrophages. Imaging was done at 0.5, 1, 1.5, and 2 h using a fluorescence microscope equipped with a 20× objective with an exposure time of 32 ms. The macrophage media with fluorescently labeled vesicles were first removed, and the cells were washed twice with 1 mL of L-15 prior to the addition of 1 mL of L-15 to the cells for imaging.

### Example 19: Cisplatin-Loaded MEVs

100 million M0, M1, or M2 cells were used to generate macrophage-derived, cisplatin-loaded vesicles and deliver them to A549 cells. Macrophage media were first aspirated off, and 3 mL of PBS was added to each flask prior to scraping them. The cell solution was collected into a 50 mL centrifuge tube, and the number of cells was determined using a hematocytometer. The cell solution was pelleted at 2000×g for 2 min at 4° C. The supernatant was discarded, and cells were resuspended in 8 mL of 8.33 mM cisplatin solution made in PBS with 1 tablet of the protease inhibitor. The cell solution was nitrogen-cavitated using a prechilled nitrogen decompressor on ice at 300 psi for 5 min. The cell lysate obtained was centrifuged at 4000×g for 10 min at 4° C. The pellet thus obtained was discarded, and the obtained supernatant was centrifuged at 10,000×g for 20 min at 4° C. The supernatant obtained was again subjected to ultracentrifugation at 100,000×g for 60 min at 4° C. to collect the pellet containing cisplatin-loaded nanovesicles. This final pellet was first washed with 1 mL of PBS twice and resuspended in 750 μL of PBS. Empty vesicles were generated using the same procedure discussed above but in the absence of cisplatin.

### Example 20: Cisplatin Concentration in MEVs

The concentration of cisplatin loaded in vesicles was determined using inductively coupled plasma-optical emission spectrometry (ICP-OES). Cisplatin-loaded MEVs were first treated with 1% Triton X-100 to dissolve the lipid bilayer, followed by 70% nitric acid treatment to release platinum from cisplatin. The resulting solution was further incubated on a heat block at 60° C. for 2 h, followed by dilution to 5 mL, such that the final nitric acid concentration was 10% for analysis using ICP-OES. A standard curve using platinum standards in 10% nitric acid solution was used to determine the concentration. Ytterbium was used as an internal standard to compensate for the internal drift of the instrument. It was previously shown that vesicles generated by nitrogen cavitation are stable with no apparent cisplatin leakage for 72 h.

### Example 21: Cytokine Analysis

MEVs were generated as described before. M1EVs were generated from 100 million M1 macrophages and resuspended in 500 μL of PBS. The number of vesicles present in the resuspension was determined using NTA. 5.49×10⁹ M1EVs were added into each well of a 24-well plate containing 1 million M0 and M2 macrophages in 950 μL of replating media. The plate was left to incubate at 37° C. for 24 h. After 24 h of incubation, MCM were collected in an Eppendorf tube (1 mL) and later used for pro-inflammatory cytokine analysis. M2EVs were generated as before using M2 macrophages. 7.6×10⁹ M2EVs were added to each well containing M0 and M1 macrophages. The plate was left to incubate at 37° C. for 24 h before collecting the media for cytokine analysis. A mouse pro-inflammatory sevenplex assay was performed, following the manufacturer's protocol. Briefly, 25 μL of calibrators and MCM were added to each well of a capture antibody-precoated MSD well plate. The plate was then allowed to incubate for an hour, and the detection antibody was added into each well of the MSD. After vigorously shaking the plate for an hour, it was then washed with 0.5% tween PBS. The Read buffer was finally added to each well and analyzed on the MESO SECTOR imager from Meso Scale Discovery. Standard curves were obtained by fitting the electrochemiluminescence signal from calibrators using Meso Scale Delivery Workbench analysis software.

### Example 22: In Vivo Delivery

A549 cells (1×10⁶) were injected subcutaneously into the interscapular region of 6-week-old athymic nude mice. The mice were monitored until palpable xenograft tumors developed greater than 200 mm3. M1EVs were generated using 100 million M1 macrophages by the procedure mentioned above. A NanoSight 300 multiple particle tracking system was used to determine the mean diameter and the concentration of MEVs. M1EVs were then labeled with the DiR near-infrared fluorescent dye. Briefly, 1 μL of 1 mM DiR was added to 199 μL of the vesicle resuspension so that the final concentration of DiR in the vesicle resuspension was 5 μM. DiR-labeled vesicles were separated from free DiR using a size exclusion PD MidiTrap column equilibrated with PBS. 100 μL of DiR-labeled M1EVs was then injected into the lateral tail vein of tumor-bearing mice. Isoflurane gas was used to anesthetize mice for imaging 72 h post injection using an IVIS Spectrum In vivo Imaging System (PerkinElmer) controlled with Livinglmage software (PerkinElmer). Epifluorescence images were obtained using 710 nm excitation and 760 nm emission filters, f/stop number 4 and binning factor 4, with a 35 s exposure.

### Example 23: Characterization of Cell-Derived Vesicles

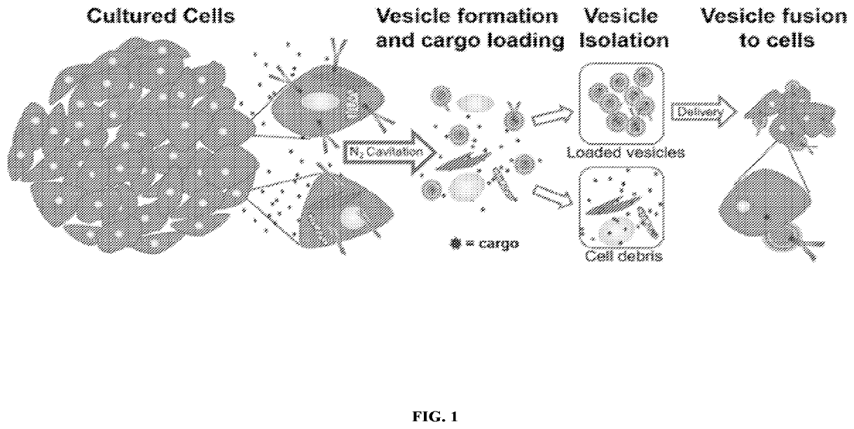

Vesicles were generated through nitrogen cavitation where cells in solution are subjected to high-pressure N2. The pressure is rapidly released resulting in the formation of gas bubbles that fragment the cellular membranes. These small fragments then reform to generate enclosed vesicles. Vesicles were separated from the remaining cell debris through a series of centrifugation steps. A schematic of the vesicle generation and isolation process is shown in FIG. 1. One advantage of this approach is that the solution containing the cells during cavitation is encapsulated in the vesicles. Thus, therapeutics or other cargos are entrapped in the vesicles with high efficiency at the time of vesicle formation.

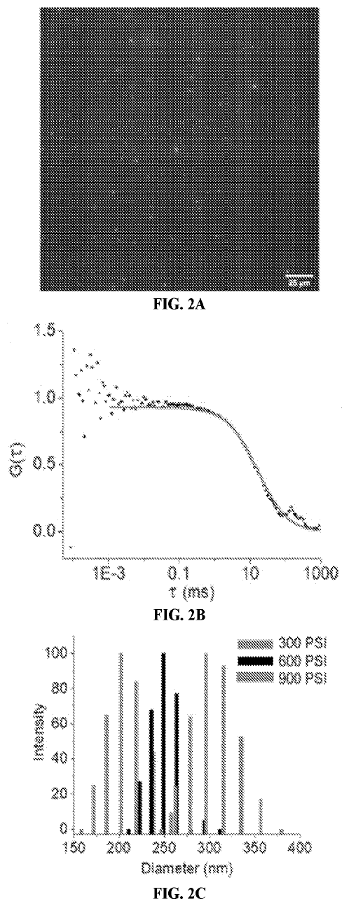

An image of vesicles generated using nitrogen cavitation from human embryonic kidney 293 cells (HEK) formed in the presence of fluorescein, a fluorescent dye, is shown in FIG. 2A. The fluorescence image shows punctate regions indicating that the fluorophore is trapped inside the vesicles verifying encapsulation. The cargo, encapsulated by a phospholipid bilayer, is safeguarded from free solution. To illustrate the versatility of this approach a series of studies were performed using vesicles from HEK, human colorectal cancer (HCT 116), human lung cancer (A549), and macrophage-like cell lines (RAW 264.7).

The collection of vesicles endogenously expressed by cells, such as exosomes, suffers from relatively poor yields. To determine the yield of the preparation, fluorescence correlation spectroscopy (FCS) was performed. Vesicles were generated from approximately 40 million A549 cells in culture using nitrogen cavitation. After the vesicles were isolated, they were labeled through the incorporation of a lipophilic dialkylcarbocyanine fluorophore (DiI), which is nonfluorescent in an aqueous solution but emits brightly when embedded in a lipid bilayer. FCS tracks fluctuations in fluorescence as vesicles diffuse through the focal volume. Both the diffusion time and the average number of molecules can be extracted from the autocorrelation curve (FIG. 2B). The FCS focal volume was calibrated using commercial tetraspeck beads leading to a determination that the preparations yielded ˜4−10¹¹ vesicles per mL, which is approximately 1.3×10¹¹ vesicles per preparation (40 million cells). Thus, a relatively large number of vesicles were able to be generated from a modest number of cells.

To further characterize cell-derived vesicles, dynamic light scattering (DLS) was performed to determine the diameter of vesicles generated via nitrogen cavitation. FIG. 2C shows the distribution of vesicle diameters of a typical preparation at different pressures. The 200 nm observed at 300 psi is slightly larger than standard exosomes (100 to 150 nm) but is within a similar range that is unlikely to affect cell delivery. To determine stability, the surface charge (zeta potential) of cell-derived vesicles suspended in a PBS buffer was also measured. Vesicles preparations exhibited a surface charge of −2.5 mV. DLS measurements of vesicles in solution after 6 h showed no changes in size distributions over time. To determine how pressure influenced the diameter of vesicles, vesicles were generated with nitrogen cavitation pressures of 300 (red), 600 (black), and 900 psi (blue) (FIG. 2C). Interestingly, 300 psi yields the smallest vesicles whereas 900 psi yielded the largest. Vesicle size was clearly dependent on the cavitation pressure. In addition to yielding tunable vesicle diameters, one advantage of cavitation over other techniques to fracture the membrane is that it does not generate heat that can damage samples or alter the chemical composition of the cell medium. This results in the formation of relatively uniform vesicles likely due to all the cells in solution being exposed to the same pressure conditions.

### Example 24: Determining Vesicle Targeting Specificity Across Different Cell Types

Previous studies have shown that exosomes and vesicles generated from cancer cells preferentially target the same cell type from which they were derived. To determine the degree of targeting specificity of vesicles generated via nitrogen across cell types, a series of studies were performed, comparing the delivery of labeled vesicles to the cell type from which the vesicles originated versus alternate cell types.

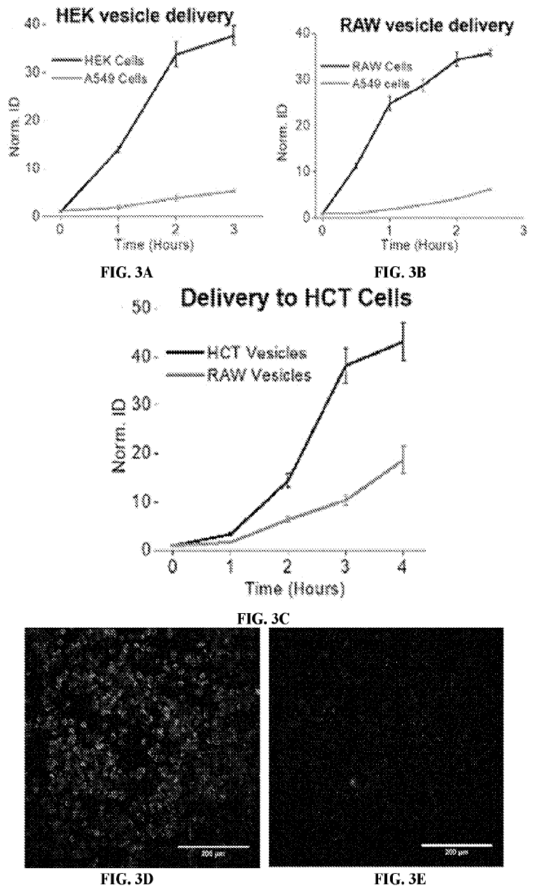

Vesicles from HEK cells were generated and they were labeled with DiI. The efficiency of delivery to both HEK and A549 cells was determined by measuring the fluorescence signal at time points over 4 h. 5×10⁹ vesicles were added to each cell culture condition and allowed them to incubate with the cells. Vesicles were then rinsed from the cells, and the cells were subsequently imaged using wide-field microscopy. Most cell types showed a clear targeting specificity for the cell type where they originated. At the 2 h time point of HEK vesicle delivery, HEK cells exhibited ˜10 times as much fluorescence as A549 cells after incubation with the same number of vesicles for the same time period (FIG. 3A). Similarly, RAW vesicles were much more efficiently delivered (8×) to RAW cells as they were to A549 cells (FIG. 3B).

The delivery of different vesicles to the same cell type was also compared. While HCT vesicles were approximately 3 times more efficient at delivering cargo to HCT cells as compared to RAW vesicles, the RAW vesicles still exhibited targeting properties for the HCT cells (FIG. 3C). Combined results show that vesicles tend to have an affinity for the delivery of cargo to the same cell type from which they originated.

The wide-field image comparing RAW vesicle delivery to RAW cells versus delivery to A549 cells after 2.5 h (FIG. 3D, 3E) shows a clear preference for delivery to RAW cells. However, the ability of RAW vesicles to effectively target cancer cells with only a 3-fold deficit as compared to cancer vesicles could allow macrophage vesicles to be used as a general delivery vehicle.

The use of cancer vesicles for clinical application is limited due to the likelihood for them to increase the metastatic potential in vivo. Additionally, the tumor microenvironment consists of a heterogenous mixture of cells including large numbers of macrophages. Thus, macrophages offer better long-term potential for clinical applications. These studies illustrate the ability of nanoscale cell-derived vesicles to deliver lipophilic cargo to cells.

### Example 25: Determining In Vitro Efficacy for Therapeutic Delivery

The efficient delivery of therapeutics to the interior of the cell is one of the primary challenges of cell delivery vehicles. A set of experiments was performed to measure the efficiency of drug loading into cell-derived vesicles and then the efficiency of delivery to cancer cells. Vesicles were generated from A549 cells in the presence of 8.33 mM cisplatin. To verify that the vesicles successfully encapsulated cisplatin, the drug concentration in the vesicles was measured directly after formation and separation from free cisplatin. The cisplatin in vesicles as measured by ICP-OES for 1.3×10¹¹ vesicles was ˜3 μg of total cisplatin. This effectively shows that the vesicles generated from nitrogen cavitation can encapsulate cisplatin at the time of formation, protecting it from the environment outside of the vesicle. To further verify that stability of encapsulation over time, the same ICP-OES studies were performed at intervals over 3 days after drug encapsulation on the supernatant to measure any cisplatin leakage from the vesicles. Vesicles maintain roughly the same concentration at each time point showing the stability of encapsulation with a total decrease of 23 ng of cisplatin from day 1 through day 3. Cell-derived vesicles are able to efficiently encapsulate a large concentration of chemotherapeutics and remain stable over 2 days with no apparent leakage. Cell delivery experiments were then performed to determine if drug-loaded vesicles could be used for therapeutic delivery by comparing the efficacy of free cisplatin to that of cisplatin encapsulated in vesicles.

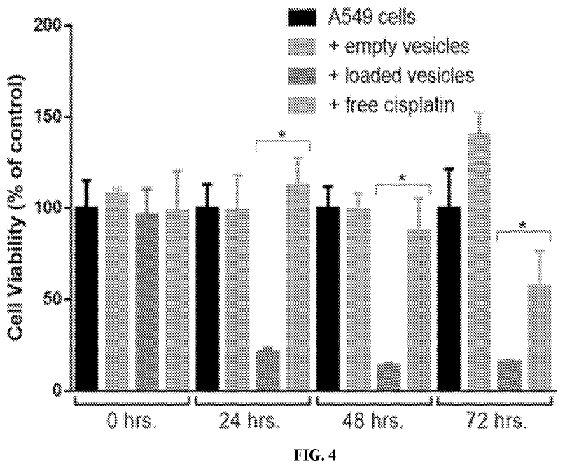

A control study was performed to determine whether the vesicles themselves had any effect on cell growth. Unloaded A549-derived vesicles were generated and delivered them to A549 cells. Cell viability was determined using a fluorescence assay (alamar blue), where the fluorescence intensity scales with the population of live cells. Comparing cell growth and viability to that of untreated cells, empty vesicles appeared to have no effect on cell growth (FIG. 4). With a clear indication that vesicles targeted the same cells from which they originated (FIG. 3A-3C) and that vesicles did not alter cell viability, the effect of free cisplatin and cisplatin-loaded vesicles on A549 cell proliferation was compared. Cisplatin was selected because of its hydrophilic nature and because it is a first-line therapeutic for lung cancer. Vesicles were loaded through nitrogen cavitation in the presence of cisplatin (8.33 mM). Vesicles were then added to A549 cells in culture, and the cell proliferation was compared to cells alone and those in the presence of free cisplatin in solution at the same levels (3 μg) as were measured in the vesicle solution. This low concentration of free cisplatin resulted in no apparent cell death at 24 h while cisplatin-loaded vesicles resulted in 70% cell death. By the 48 h time point, loaded cell-derived vesicles resulted in 90% cell death, while free cisplatin resulted in 15% cell death. At 72 h, free cisplatin led to 50% cell death while vesicles maintained approximately 90% cell death. Vesicles (1×10¹⁰) containing 0.3 μg of cisplatin (˜4 μM) delivered to A549 cells were much more effective at limiting cell viability than the same concentration of free cisplatin. This verified the capability of the vesicles to more efficiently deliver the drug across the cell membrane.

### Example 26: Cell Derived Vesicles for Gene Delivery

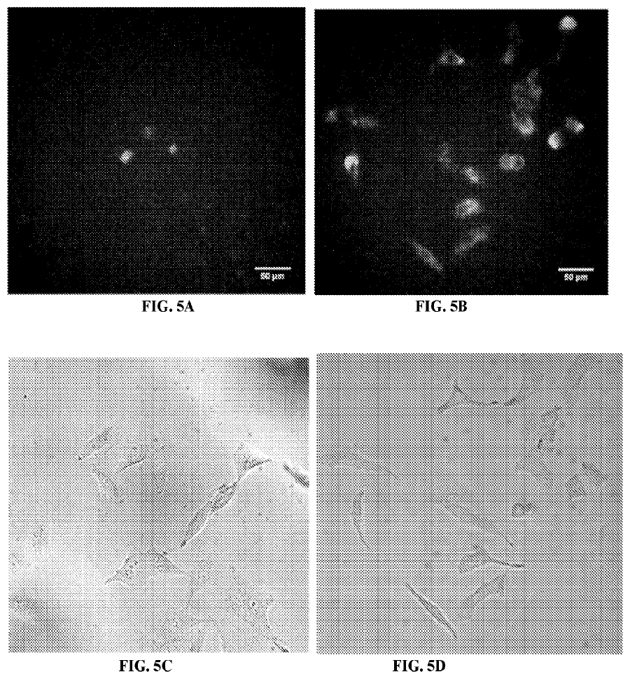

To demonstrate the versatility of vesicle-based delivery, plasmid DNA was also encapsulated for gene delivery. Current strategies for gene delivery primarily use transfection reagents such as cationic lipids for efficient delivery. Vesicles were generated in the presence of a plasmid that encoded for the fluorescent protein Dendra2. Vesicles were loaded via the same nitrogen cavitation approach and then incubated with HEK cells for 48 h. The cells were then visualized using wide-field fluorescence microscopy to identify cells expressing the fluorescent protein. Virtually none of the control cells showed any fluorescence. The two cells exhibiting any fluorescence (FIG. 5A) in the control sample correspond to autofluorescence from dead cells in the field of view and the rest of the live cells show no fluorescence as indicated in the bright-field image shown in the Supporting Information (FIG. 5C). The majority of HEK cells in the vesicle-treated sample had taken up the plasmid and produced a characteristic green fluorescence. The bright-field image is shown in Supporting Information (FIG. 5D). Here ˜1×10¹⁰ vesicles were used for delivery. As can be seen from the similar level of fluorescence across most cells, the delivery in culture and loading of vesicles appeared to be homogenous across the sample. This verifies that vesicles are capable of delivering DNA across the cell membrane to the interior of the cell.

### Example 27: Delivery of Hydrophilic Cargo

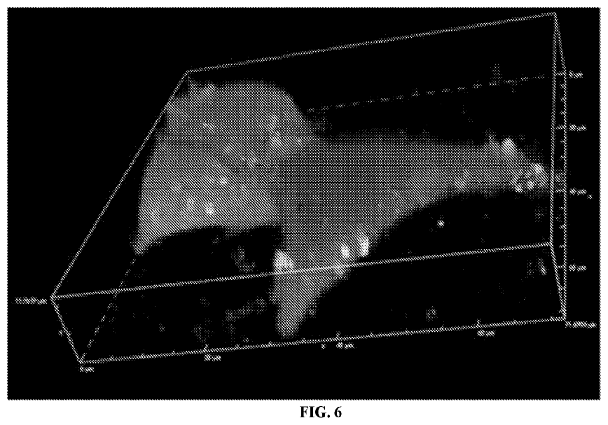

To visualize the delivery of cargo to the cell interior, vesicles were generated while simultaneously encapsulating the green emitting fluorophore, fluorescein. After generating the vesicles, the vesicle membrane was labeled with DiD, a lipophilic dye. This allowed for observation of the delivery of cargo within the vesicles to the interior of the cell as well as observe integration of the vesicle membrane into the membrane of the target cells. After 45 min of exposure, vesicles were rinsed from the cells and confocal imaging was performed. Clear fluorescein fluorescence permeated the interior of the cell indicating its presence in the cytosol (FIG. 6). At the same time, isolated vesicles were observed on the cell surface. The presence of fluorescein in the cytosol after only 45 min verifies the encapsulation and delivery of hydrophilic cargo. Individual vesicles labeled with DiD were observed on the cell surface.

### Example 28: In Vivo Targeting and Delivery of Vesicles

A set of studies were also performed to determine if cell-derived vesicles could be used to target a specific tissue in a live animal. Vesicles were generated from HEK cells, A549 cells, and RAW 264.7 cells and labeled each separately with DiR to enable tracking to specific sites within the animal. RAW cells were used because the tumor environment contains large numbers of macrophages, RAW vesicles showed some targeting affinity for cancer cells, and targeting delivery with cancer cell-derived vesicles is not feasible for clinical applications. There is concern for the potential of cancer vesicles to lead to an increase in the spread of cancer throughout the body.

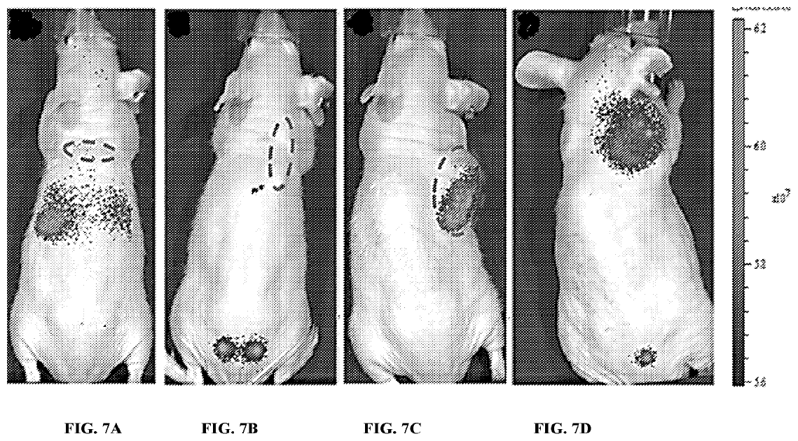

A male outbred athymic nude (nu/nu) mouse from Jackson Laboratories implanted with a subcutaneous tumor xenograft composed of A549 or HCT116 cells was used. After the tumor reached at least 100 mm³, the labeled vesicles (˜2×10¹¹) were injected, systemically, through the tail vein. In vivo imaging was performed 24 h postinjection using an IVIS whole animal imager. Injection of DiR alone lead to nonspecific accumulation, while HEK vesicles accumulated in the area of the bladder (FIG. 7A, 7B). Clear fluorescence was observed in the tumor site for the delivery of A549 and RAW vesicles (FIG. 7C, 7D). These experiments verify the in vivo targeting capability of both A549 vesicles and RAW vesicles for the tumor xenograft.

These studies demonstrate that cell-derived vesicles can be efficiently loaded with a wide variety of cargos. They also exhibit similar properties as exosomes in that they specifically target the cell from which they originated. However, cell-derived vesicles can be easily loaded and generated at higher yields than exosomes. These properties can be harnessed to efficiently deliver cargos ranging from therapeutics to DNA across the cell membrane. These vesicles demonstrated targeting efficiency in animals as shown through their delivery to tumor sites. Overall, cell-derived vesicles can be used as a versatile cell delivery vehicle both in vitro and in vivo.

### Example 29: Characterization of MEVs

MEVs are generated through mechanical disruption of the cell membrane into nano-sized fragments which reform into vesicles. Here, a prechilled nitrogen decompressor was used and maintained BMDMs at a pressure of 300 psi for at least 5 min. The sudden release of pressure causes the cell membrane to fragment, and because the phospholipids composing the membrane are amphipathic, the hydrophobic effect drives these fragments to spontaneously form vesicles in aqueous solutions. These vesicles are separated from cellular debris by a series of centrifugation and ultracentrifugation steps as depicted in FIG. 8.

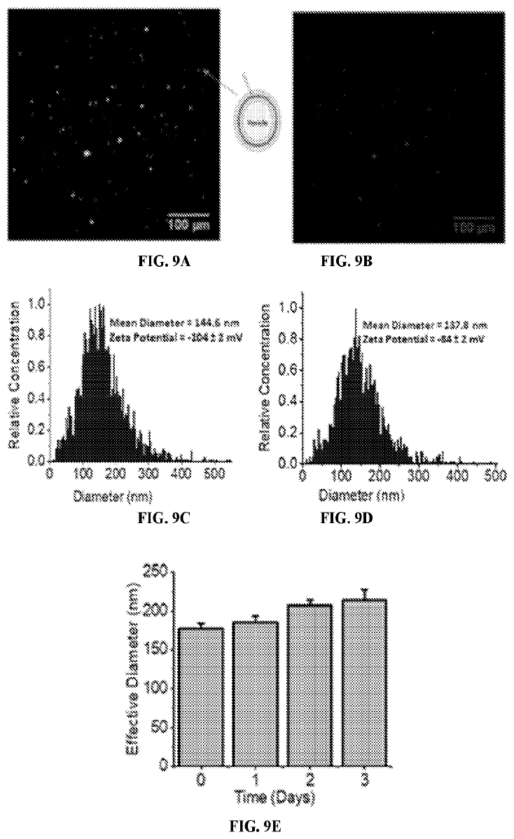

Vesicles are generated in the presence of the solution in which the cells were initially suspended, leading to the encapsulation of any hydrophilic therapeutic or other cargo present in the aqueous solution during vesicle generation. FIG. 9A shows a fluorescence image of MEVs generated by nitrogen cavitation in the presence of a fluorescein-containing solution. Fluorescein is a fluorescent dye that is soluble in an aqueous medium and is entrapped within the vesicles during their formation. Green punctate regions in the fluorescence image indicate the presence of fluorescein inside the vesicles and the successful loading of cargo during vesicle generation. Similarly, MEVs can be labeled with a lipophilic dye such as DiI. The fluorescence image in FIG. 9B shows red punctate regions corresponding to DiI incorporation into the vesicle membrane.

To determine the yield of MEVs during nitrogen cavitation, multiple particle tracking was performed to extract both the size distribution of vesicles and their concentration. Particle tracking (Nanosight 300) determines particle size based on diffusion rates and the concentration by counting the number of particles in a defined volume. Vesicles generated from approximately 100 million M1 BMDMs in culture using nitrogen cavitation yielded 5.5×10¹⁰ vesicles (M1EVs). Similarly, 100 million M2 macrophages yielded 6.9×10¹⁰ vesicles (M2EVs). The size distribution of MEVs generated by nitrogen cavitation at 300 psi is primarily between 100-200 nm, which is similar to that of exosomes. The mean diameter of M1EVs was found to be 144.6 nm (FIG. 9C) and that of M2EVs was found to be 137.8 nm (FIG. 9D). The zeta potential of MEVs suspended in the PBS buffer was measured and it was found that M1EVs had a zeta potential value of −104±2 mV and M2EVs had a zeta potential of −84±2 mV. A large negative value for the zeta potential indicates the stability of MEVs in aqueous solution. These initial characterization studies show that vesicles from BMDMs can be generated with a similar size to exosomes. Additionally, a large number of vesicles were able to be produced from a relatively small volume of tissue culture without the need to wait for long periods of time for the production of EEVs through normal physiological processes.

The stability of MEVs were tested over time to determine their potential suitability as a drug delivery vehicle where they would be required to circulate within the human body for a period of time before delivery of cargo to a specific site. The stability of MEVs generated by nitrogen cavitation were tested by incubating them in solution for 3 consecutive days. Vesicle size was monitored over time to determine the extent of aggregation. The size of MEVs remained relatively constant for the first 2 days, signifying the stability of MEVs over this interval. After 48 h, the stability gradually decreased, as shown by the increase in the size of the vesicles (FIG. 9E). Thus, in addition to their high yields, MEVs are also stable for times compatible with the likely circulation time needed for therapeutic delivery.

### Example 30: MEV Delivery to Macrophages

Previous studies have shown that vesicles generated from cellular membranes can be used as efficient therapeutic delivery vehicles to deliver cargo to the interior of the cell. In order to investigate the ability of MEVs to deliver cargo into the interior of macrophages, MEVs were generated from BMDMs stimulated to be M1 (INF-γ+LPS) and loaded with fluorescein. The M1EVs were labeled concomitantly with the lipophilic dialkylcarbocyanine fluorescent dye, DiI, which embeds into the lipid bilayer of the vesicles. Both fluorescent labels were separated from the unloaded dye using a size exclusion column.

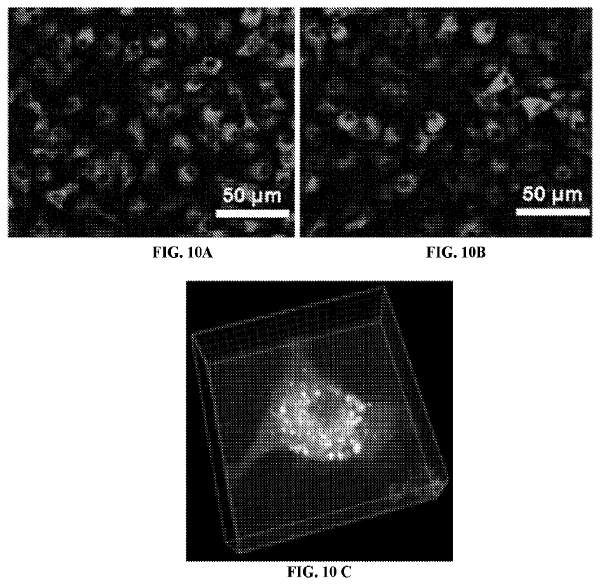

BMDMs stimulated with IL-4 were incubated, to generate M2 cells, with the M1EVs. After incubation with these M2 macrophages, bright fluorescence was observed after 2 h when imaged with confocal microscopy under both 488 nm (fluorescein) and 532 nm (DiI) excitation (FIGS. 10A and 10B). M1EVs were evident inside of M2 macrophages, as shown from the fluorescence puncta both inside and on the membrane of macrophages (FIG. 10C). At 2 h after incubation, most vesicles remain intact and isolated on the membrane as well as inside of the cell.

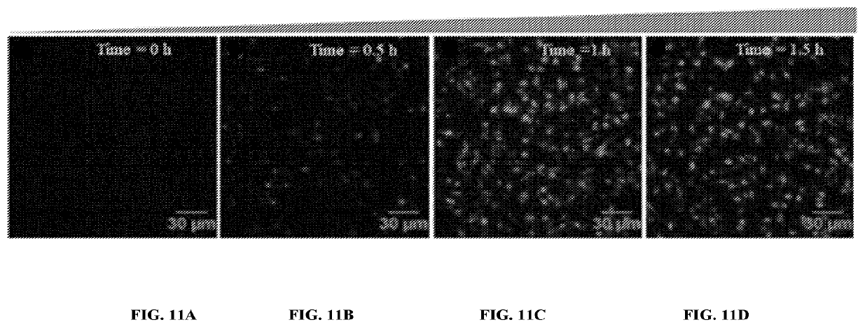

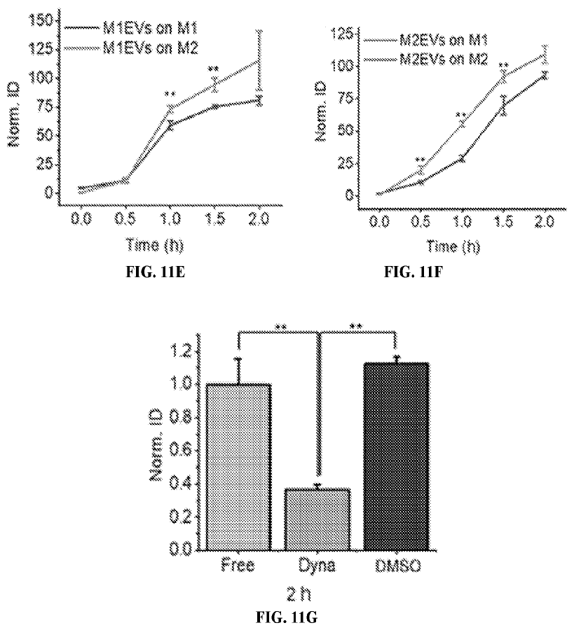

After confirming the delivery of M1EVs onto M2 macrophages, a set of experiments were performed to determine if vesicles generated from M1 and M2 BMDMs possess different macrophage targeting capabilities. DiI-labeled vesicles were generated from an equal number of M1 or M2 macrophages. The efficiency of delivery to M1 and M2 macrophages was determined by measuring the fluorescence signal at various time points over 2 h. M1EVs and M2EVs were added separately to M1 or M2-stimulated BMDMs. Vesicles were then rinsed from the cells, and the cells were subsequently imaged using wide-field microscopy. Time-dependent uptake of MEVs by macrophages was observed (FIG. 11A-11D). While both M1EVs and M2EVs were efficiently delivered to M1 and M2 macrophages, M2 macrophages showed a higher uptake of both M1EVs and M2EVs compared to M1 macrophages (FIG. 12E, 12F).

Dynamin activity is an integral component of both endocytosis and phagocytosis. Dynasore, a dynamin inhibitor, has been widely used to study the process of internalization of exosomes from the surface of the macrophage. Recent studies showed that the knockdown of dynamin 2 almost completely inhibited the uptake of exosomes by RAW264.7 macrophage-like cells. Since MEVs mimic exosomes, studies were conducted to investigate whether they exhibited a similar mechanism of vesicle internalization by macrophages. The uptake of fluorescently labeled M1EVs by M2 BMDMs was compared in the presence and absence of dynasore. Dynasore (80 μM) was added to cultured macrophages 20 min prior to the addition of labeled vesicles. M2 macrophages were left to incubate with M1EVs for 2 h and subsequently imaged by wide-field microscopy. Dynasore had no effect on the cell viability and macrophages looked morphologically similar with and without treatment. The integrated density of the fluorescence signal was calculated to compare the uptake of M1EVs by M2 macrophages. Dynasore resulted in 64% reduction in uptake of vesicles relative to the control (FIG. 11G). Similar vesicle uptake control experiments were performed in the presence of the vehicle, DMSO, at an equal concentration. There was no significant effect of DMSO on the M1EV uptake process by M2 macrophages relative to the control with no DMSO or dynasore. Macrophages are well-established phagocytotic cells. The loss of cellular uptake with dynamin inhibition coupled with the observation of intact vesicles inside macrophages indicates that macrophages are likely internalizing vesicles via phagocytosis. These results demonstrate that MEVs exhibit similar properties to exosomes and are able to target macrophages.

### Example 31: MEVs Reprogram Macrophage Phenotypes

Previous studies have shown that exosomes generated from M1 or M2 macrophages can be used to differentiate naive macrophages into the corresponding pro- or anti-inflammatory phenotypes. After confirming that MEVs can be delivered to macrophages, their ability to differentiate naive (M0) macrophages was tested. M1EVs were generated using nitrogen cavitation from cultured M1 macrophages and then delivered to M0 macrophages to compare cytokine production from M0 macrophages, M1 macrophages, and M0 macrophages incubated with M1EVs. Macrophage-conditioned media (MCM) were extracted from the cell culture of each sample. A meso-scale delivery Sevenplex ELISA was performed, which simultaneously tested for seven mouse pro-inflammatory cytokines (IFN-γ, IL-10, IL-12p70, IL-β, IL-6, KC/GRO, and TNF-α) in the cell culture supernatant. Clear pro-inflammatory markers from M1 macrophages were observed and virtually no measurable levels for most of the cytokines in the M0 culture were observed (FIG. 12A). It was found that M1EVs can reprogram M0 macrophages toward an M1 phenotype as evidenced by the increased production of each of the pro-inflammatory cytokines (n=3/group) from undetectable to 6±6% (IFN-γ), 45±2% (IL-10), 29±1% (IL-12p70), 81±63% (IL-β), 12 ±5% (IL-6), 36±13% (KC/GRO), and 20±8% (TNF-α) of the average concentration seen for M1 macrophages (FIG. 12A). These results verified that M1EVs can stimulate M0 BMDMs toward a pro-inflammatory phenotype. A shift toward a pro-inflammatory phenotype was not observed when M2MEVs were added to M0 macrophages. The results reinforce the claim that MEVs exhibit similar properties to exosomes and can be used to polarize naive macrophages.

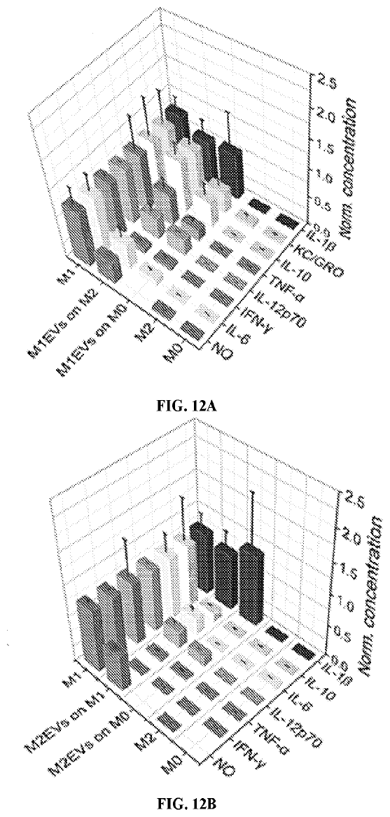

A set of experiments was performed to determine the effect of vesicle delivery on macrophages that have already been polarized toward a specific phenotype. The ability of pro-inflammatory vesicles to influence anti-inflammatory macrophages, as well as the ability of anti-inflammatory vesicles to influence pro-inflammatory macrophages, were examined. To test the capability of MEVs to reprogram already polarized macrophages, treated cultured M2 BMDMs were treated with M1EVs and compared the cytokine production from M1 macrophages, M2 macrophages, and M2 macrophages exposed to M1EVs. For M2 macrophages that had been treated with M1EVs, a significant increase was found in the production of cytokines (n=3/group) from undetectable to 10±1% (IFN-γ), 91±20% (IL-10), 37±12% (IL-12p70), 77±30% (IL-β), 44±20% (IL-6), 85±27% (KC/GRO), and 55±18% (TNF-α) of the average concentration seen for M1 macrophages (FIG. 12A). A Griess assay was performed to assess the nitric oxide (NO) presence in MCM collected from M1, M2, and M2 macrophages that were incubated with M1EVs. A significant increase in the production of nitric oxide was found from negligible initial amounts in M2 to 41±0.4% of the average concentration seen for M1 macrophages when M2 macrophages were treated with M1EVs. Comparing M2 versus M0 macrophages treated with M1EVs, M1EVs were able to induce a greater increase in pro-inflammatory indicators in M2 macrophages. Control studies showed that MEVs themselves only have marginal amounts of cytokines and they would not be responsible for the amounts seen after the shift (FIG. 13). These results indicate that M1EVs can repolarize M2 BMDMs toward a pro-inflammatory M1 phenotype as evidenced by the increase in inflammatory cytokine production.

M2 vesicles were also added to cultured M0 macrophages and compared the cytokine production from M1 macrophages, M0 macrophages, and M0 macrophages incubated with M2EVs (FIG. 12B). Upon incubation of M0 macrophages with M2EVs, M0 macrophages did not produce most of the pro-inflammatory cytokines, indicating that M2EVs do not induce most of the pro-inflammatory properties in target M0 macrophages (FIG. 12B). This indicates that the delivery of vesicles themselves does not simply generate a pro-inflammatory response that was seen only with M1EV delivery. The cytokine production from M1 macrophages, M2 macrophages, and M1 macrophages incubated with M2EVs were compared. Clear pro-inflammatory markers were observed from M1-macrophages but virtually no levels for most of the pro-inflammatory cytokines in the M2 culture. A clear decrease in the levels of all the pro-inflammatory markers was also observed for M1 macrophages that were incubated with M2EVs (FIG. 12B). M2EVs significantly attenuated cytokine released by M1 macrophages by 99% (IFN-γ), 85% (IL-10), 74% (IL-12p70), 9% (IL-β), 72% (IL-6), 78% (KC/GRO), and 96% (TNF-α) of the average concentration seen for M1 macrophages (FIG. 12B). A significant reduction (49%) in NO production by M1 macrophages that were incubated with M2EVs was observed, as compared to the average concentration seen for M1 macrophages. This indicates that M2EVs can reprogram M1 macrophages away from a pro-inflammatory phenotype. This has important implications on the use of MEVs to reprogram macrophage phenotype as part of a therapeutic approach. The phenotype used to generate MEVs appears to dictate their ability to reprogram both naive and already polarized macrophages toward a desired phenotype. The ability to alter macrophage inflammatory properties could be an important therapeutic tool to reprogram anti-inflammatory macrophages to a pro-inflammatory phenotype.

### Example 32: Repolarization of Microglia

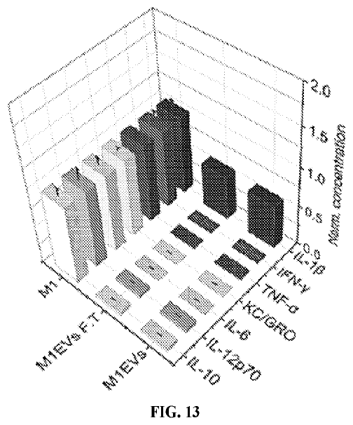

Microglia are immune cells present in the central nervous system. Similar to macrophages, microglia are also polarized to M1 and M2 phenotypes and play pro- and anti-inflammatory roles, respectively. To determine if macrophage-derived vesicles are able to reprogram microglia phenotypes, vesicles derived from macrophages were delivered to primary microglia cells in culture. M2 microglia polarization was induced using IL-4. M1EVs generated from bone marrow-derived M1 (LPS+INF-γ) macrophages were then added to cultured M2 microglia to compare the cytokine production from M1 microglia, M2 microglia, and M2 microglia incubated with M1EVs. Clear pro-inflammatory markers from M1 microglia and virtually no measurable levels for most of the cytokines were observed in the M2 microglia culture. An increase in the levels of all the pro-inflammatory markers for M2 microglia that were incubated with M1EVs was also observed (FIG. 14A-14G). The ability of M1EVs to reprogram M2-polarized microglia toward a pro-inflammatory (M1) phenotype in a controlled fashion suggests that one can reprogram both macrophage and microglia inflammatory properties by the delivery of vesicles that are targeted to specific cell types. Furthermore, similar to macrophage exosomes, MEVs can deliver the corresponding signals to unstimulated macrophages and differentiate them into specific phenotypes. This has implications for therapeutic approaches where the goal is to either initiate or suppress a pro-inflammatory response.

The ability of MEVs to reprogram immune cells is likely due to membrane-bound proteins on the surface of the vesicle. As they are derived from parent immune cells, MEVs carry a wide range of transmembrane proteins, membrane-bound cytokines, and other cell signaling endogenous ligands. These proteins can interact with membrane receptors on the target cell initiating signaling cascades that lead to repolarization.

### Example 33: Macrophage-Induced Neurotoxicity

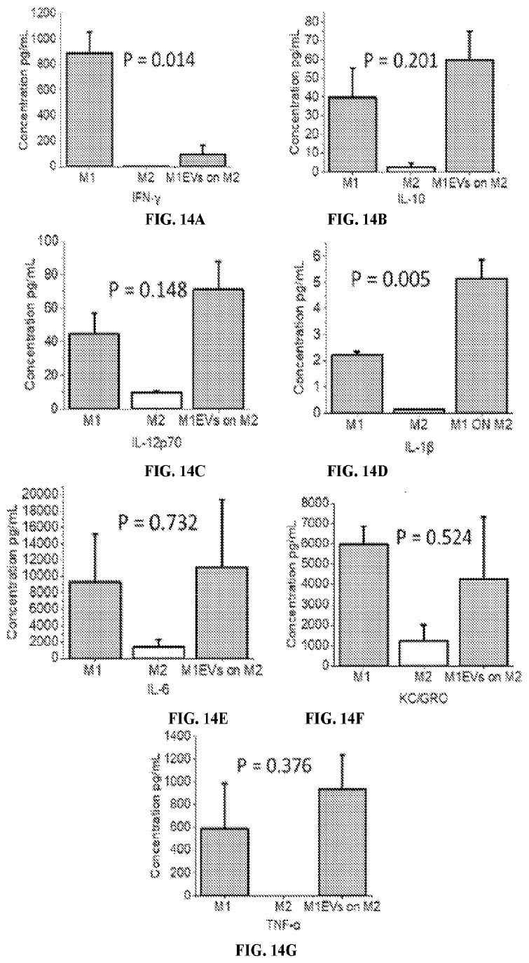

Classically activated M1 macrophages, stimulated with LPS+IFN-γ, are neurotoxic and contribute to neuronal degeneration by releasing high levels of specific pro-inflammatory cytokines and oxidative metabolites such as nitric oxides.(9,52) Pro-inflammatory cytokines such as TNF-α, IFN-γ, IL-12, and IL-6 have been found to be involved in neuronal death. Alternatively activated, M2 macrophages do not induce cell death but rather help the repair process by releasing growth factors and anti-inflammatory cytokines. Recent studies showed that azithromycin (AZM), a frequently used macrolide antibiotic, also possesses the ability to reduce macrophage-mediated neurotoxicity by altering macrophage phenotype from pro-inflammatory to anti-inflammatory.(9,56) Studies were conducted to determine if MEV-induced reprogramming of M1 macrophages toward an M2 phenotype could moderate neurotoxicity in a similar fashion to AZM.(9) LPS+IFN-γ was used to stimulate an M1 macrophage phenotype and IL-4 to stimulate an M2 phenotype. Vesicles were generated from M2 macrophages and then exposed M1 macrophages to M2 MEVs, which reduces the production of pro-inflammatory cytokines (FIG. 12B). The supernatant was colleged from M1 macrophages and M1 macrophages that had been exposed to M2EVs for 24 h. Media from both conditions were used to separately treat differentiated Neuro-2A (N2a) cells. N2a cells are a mouse neural crest-derived cell line which possess the ability to differentiate into cells with neuron-like characteristics. Media from M1 macrophages resulted in a 40% reduction in neuron viability relative to the control media (FIG. 15A) (n=5/group). Media collected from M1 macrophages that had been exposed to M2EVs for 24 h resulted in no significant reduction in neuron viability relative to the control (FIG. 15A). This is likely due to the significant reduction of pro-inflammatory cytokines by M1 macrophages upon incubation with M2EVs (comparable to AZM-treated M1 cells, FIG. 15B), and the corresponding increase in neuron viability suggests that pro-inflammatory cytokines released by M1 macrophages play a major role in the cytotoxicity of N2a cells. These results also indicate that reprogramming M1 macrophages toward an anti-inflammatory phenotype using M2EVs is comparable to an immunomodulatory pharmacological agent and reduces the cytotoxicity normally observed with pro-inflammatory macrophages.

### Example 34: MEVs for Therapeutic Delivery

Previous studies have shown that vesicles generated from A549 (lung carcinoma) cells can target as well as deliver chemotherapeutics to the same cell type from which they were generated. There is some concern about the use of cancer cell-derived vesicles for drug delivery because of the potential for these vesicles to be cleared by the body's immune system and that these vesicles might increase the metastatic potential.

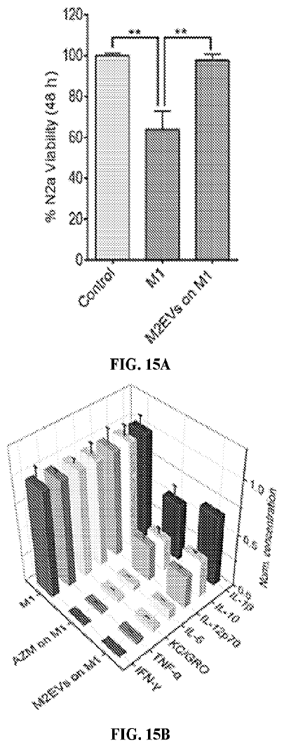

MEVs were tested to determine if they had similar targeting and therapeutic delivery features as were previously observed for cancer cell vesicles. MEVs lack any cancer characteristics and would not increase the metastatic potential. An experiment was performed to determine the targeting ability of MEVs for A549 cells. Vesicles were generated from macrophages and labeled them with DiI. The efficiency of delivery of MEVs was determined by measuring the fluorescence signal at time points over 4 h. An increase was observed in the fluorescence intensity over time resulting from an uptake of MEVs by the A549 cells. The uptake of MEVs by A549 cells suggests that MEVs can serve as a potential drug delivery vehicle in the delivery of chemotherapeutics (FIG. 16).

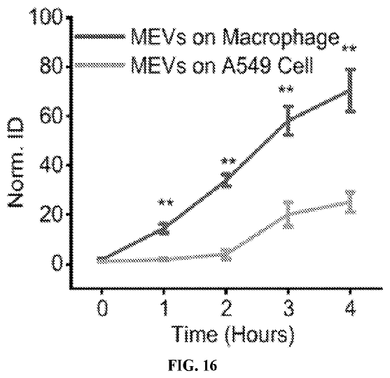

It was next determined if MEVs could be loaded with cisplatin and delivered to cancer cells while maintaining the efficacy of the therapeutic. The specificity of cisplatin delivery onto A549 cells was compared using M0EVs, M1EVs, and M2EVs (FIG. 17A). Empty M0EVs and M2EVs had no significant effect on A549 cell proliferation. However, M1EVs resulted in 10% A549 cell death in 24 h. Cisplatin-loaded M0EVs (Cs-M0EVs), M1EVs (Cs-M1EVs), and M2EVs (Cs-M2EVs) were generated from an equal number of M0, M1, and M2 macrophages. Previous studies have shown that vesicles generated using nitrogen cavitation can efficiently encapsulate chemotherapeutics and are stable for 2 days. Therapeutic-loaded MEVs were then delivered to cancer cells to determine cytotoxicity. Cisplatin-loaded M0 and M2 vesicles resulted in 45 and 40% cell death, respectively, at 24 h. However, cisplatin-loaded M1 vesicles resulted in a 60% A549 cell death in 24 h (FIG. 17A). This is a clear indication that cisplatin-loaded M1 MEVs are more efficient in killing cancer cells compared to cisplatin-loaded M0 and cisplatin-loaded M2 macrophages. Furthermore, with reference, FIG. 17B (and also with reference to FIG. 4), cisplatin loaded MEVs are more effective than cisplatin alone in lung and ovarian models.

### Example 35: In Vivo Delivery of MEVs to Tumor Xenografts

To determine if MEVs exhibited similar targeting features in vivo as observed in cell culture, vesicles were generated from M1 macrophages and labeled them with a membrane dye, DiR. The free dye was separated from MEVs using PD Miniprep columns. M1EVs were found to target tumor xenografts (subcutaneous injection A549 cells) implanted in immune-compromised athymic nude (nu/nu) mice. After the tumor xenograft reached at least 100 mm3, 2×10¹⁰ vesicles were injected through the tail vein of each of the three different mice. An IVIS whole animal imager was used for in vivo imaging. Imaging was done at 48 and 72 h post injection of labeled vesicles. DiR alone when injected into the mice as a control showed nonspecific accumulation. Clear delivery of the labeled vesicles to the tumor xenograft was observed at 72 h post injection of labeled vesicles (FIG. 17C). These results verify that M1EVs can specifically target the tumor tissue in vivo.

### Example 36: MEVs Repolarization of Macrophages and Anticancer Activity

Human peripheral blood mononuclear cell-derived monocytes were cultured with M-CSF (50 ng/mL) for 6 days. M0 macrophages were stimulated for 24 hours with LPS (20 ng/mL) plus human IFN-γ (20 ng/mL) for M1 macrophages, or with human IL-4 (20 ng/mL) plus human IL-13 (20 ng/mL) for M2 macrophages.

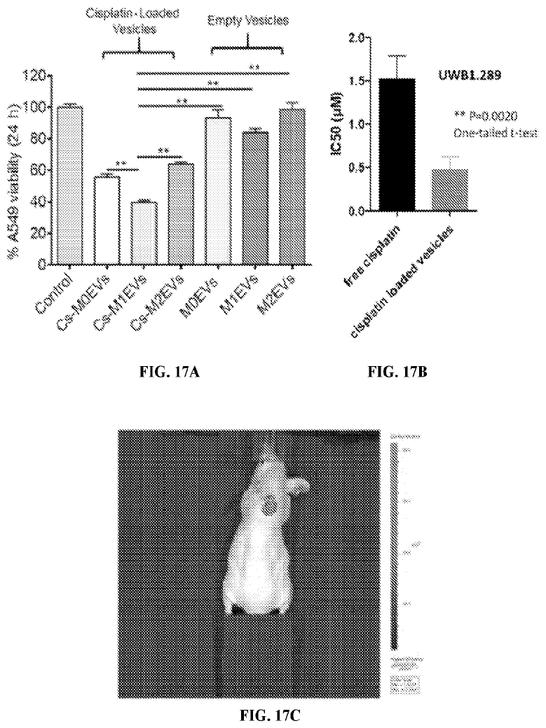

With reference to FIG. 18A, M2 macrophages were treated with M1 MEVs for 24 hours. Cell supernatants were collected after 24 hours and assayed in duplicate using a human TNF-α Quantikine ELISA kit (R&D Systems). Based on quantification of the TNFa released by the macrophages the M2 macrophages that were incubated with M1EVs repolarized toward an M1 phenotype.

With reference to FIGS. 18B and 18C, human ovarian cancer cells (Caov-3) were plated with M2 macrophages. 20% or 10% dilution of human M1 MEVS was added to Caov-3 cells only and Caov-3 plus M2 cells in duplicate and treated for 24 hours. Cell viability was measured at 96 hrs using a cell proliferation assay. The percent viability of Caov-3 cells alone (FIG. 18B) compared to M2 macrophages plus Caov-3 cells (FIG. 18C), which were treated with 20% or 10% M1 MEVs for 24 hrs, indicate that the MEVs have anticancer activity.

### Example 37: Ability of MEVs to Repolarize Human Macrophages is Independent of MEV Source

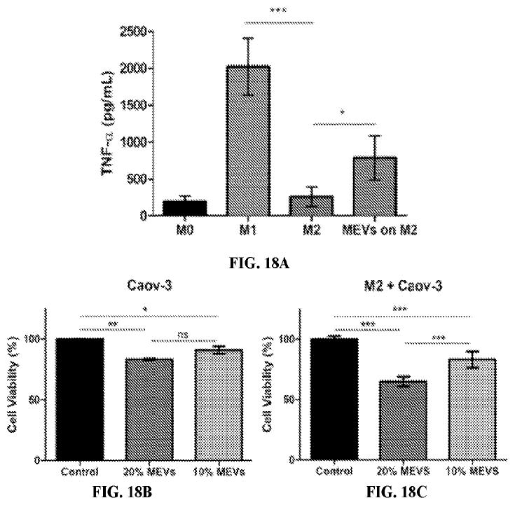

Repolarization of M2 macrophages was validated by analysis of cytokines, RNA, and surface markers (FIG. 19A). M1 MEVs derived from RAW cells, mouse bone marrow-derived cells, and human PBMC-derived macrophages are able to re-polarize M2 macrophages. With reference to FIG. 19 B, human macrophages repolarized using human M1 MEVs show significant shifts in TNFα secretion. Human M0, M1, and M2 macrophages were generated as previously described. Cell supernatants were collected at 24 hrs (24) after initial polarization and 24 hrs after adding MEVs (48). Supernatants from M1 macrophage flasks used for MEV generation were also collected 24 hrs following polarization (F1) to validate M1 macrophage polarization. Data were analyzed using PRISM 9 software by indicated statistical tests. At 24 hrs, all M1 populations were significantly higher compared to other macrophage populations for TNFα secretion (#, p<0.0001, One-Way ANOVA). M0 and M2 MEV treated populations showed significantly higher secretion of TNFα compared to untreated M0 and M2 at 48 hrs following MEV treatment (%, p<0.01**, Student's unpaired t-test).

### Example 38: MEVs Contain Similar Surface Proteins as Their Parent Macrophage, and Specific Exosomal-Marker Proteins

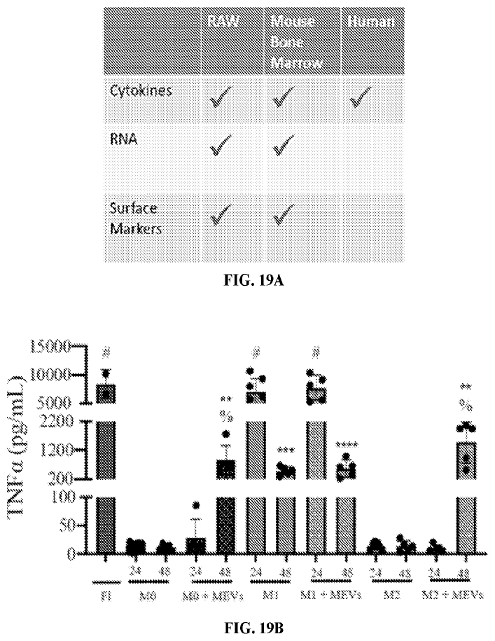

Macrophages have surface proteins, which can be indicative of phenotype and source. FIG. 20 is a schematic illustration showing exemplary markers associated with assessing M1 MEV surface proteins repolarizing M2 macrophages.

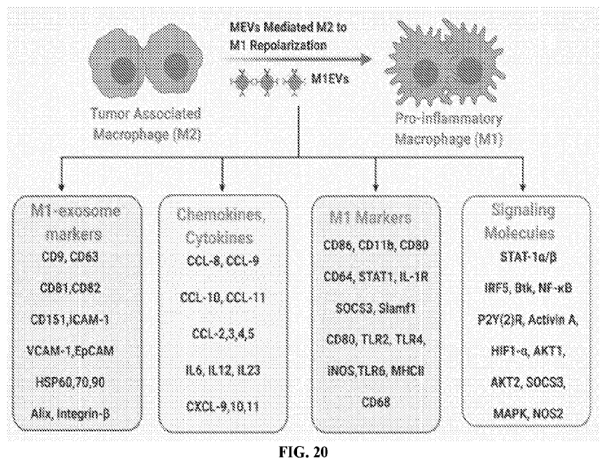

With reference to FIG. 21, cultured cells or MEVs were lysed and proteins were denatured and separated on a gel and stained for antibodies specific to the listed protein. As indicated by the macrophage membrane marker proteins on M1EVs, MEVs contain similar surface proteins as their parent macrophage.

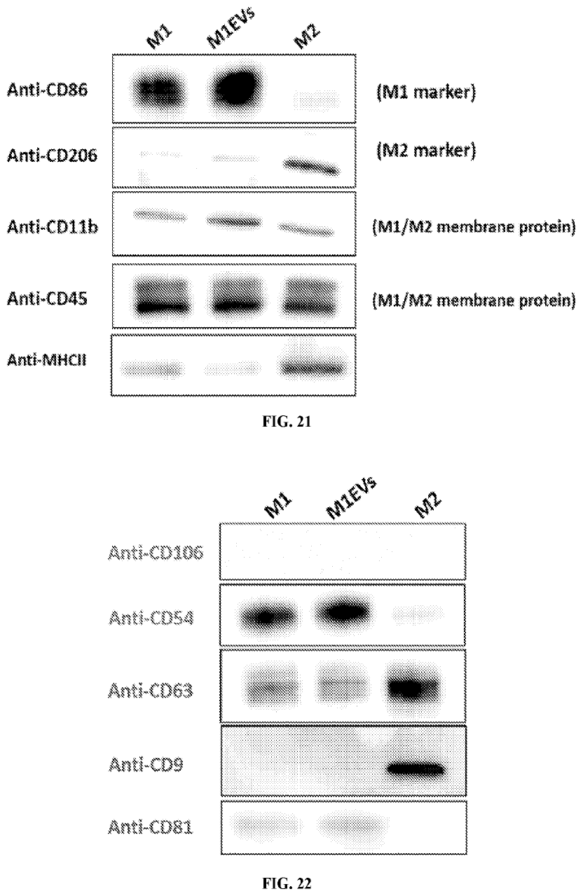

With reference to FIG. 22, Cultured cells or MEVs were lysed and proteins were denatured and separated on a gel and stained for antibodies specific to the listed protein. This was used to identify specific exosomal-marker proteins on M1EVs. These data suggest that ICAM1/CD54 on M1 MEVs could suppress MCP1 via miR-024 on M2 macrophages. these data also show expression of CD9,CD63, and CD81, which are transmembrane proteins that play a role in activation.

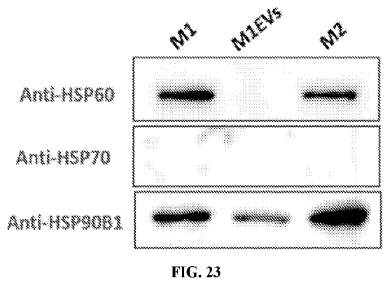

With reference to FIG. 23, cultured cells or MEVs were lysed and proteins were denatured and separated on a gel and stained for antibodies specific to the listed protein. This was used to identify specific heat shock proteins on M1EVs. Some of the heat shock proteins like HSP60, HSP70 are cytoplasmic while others like HSP90B1(gp96) are found in endoplasmic reticulum. HSP60, HSP70, and HSP90B1 have all been implicated in macrophage activation. As the results show, M1EVs only contain HSP90B1. HSP90B1 is a chaperone for Toll Like Receptors.

The data included herein establish that BMDMs can be used to engineer nano-sized vesicles with high yield using nitrogen cavitation. These vesicles can be loaded with various cargo during their generation and can be used as drug delivery vehicles both in vitro and in vivo. In addition, MEVs when interacting with the macrophage itself possess the ability to reprogram macrophages and microglia into specific inflammatory phenotypes that dictate the macrophage function (e.g., neurotoxicity and tumor migration). This shows the utility of MEVs as versatile therapeutics to target and reprogram macrophages.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Akbarzadeh, A.; Rezaei-Sadabady, R.; Davaran, S.; Joo, S. W.; Zarghami, N.; Hanifehpour, Y.; Samiei, M.; Kouhi, M.; Nejati-Koshki, K Liposome: classification, preparation, and applications. Nanoscale Res. Lett. 2013, 8, 102-102.
2. Allen, T. M. Long-circulating (sterically stabilized) liposomes for targeted drug delivery. Trends Pharmacol. Sci. 1994, 15, 215-20.
3. Andreu, Z. and M. Yáñez-Mó, Tetraspanins in Extracellular Vesicle Formation and Function. Frontiers in Immunology, 2014. 5: p. 442.
4. Anestakis, D.; Petanidis, S.; Kalyvas, S.; Nday, C.; Tsave, 0.; Kioseoglou, E.; Salifoglou, A. Mechanisms and applications of interleukins in Cancer Immunotherapy. Int. J. Mol. Sci. 2015, 16, 1691-1710.
5. Antimisiaris, S. G.; Mourtas, S.; Marazioti, A. Exosomes and Exosome-Inspired Vesicles for Targeted Drug Delivery. Pharmaceutics 2018, 10, 218.
6. Aston, W. J., et al., A systematic investigation of the maximum tolerated dose of cytotoxic chemotherapy with and without supportive care in mice. BMC Cancer, 2017. 17(1): p. 684.
7. Bate, C.; Kempster, S. L.; Last, V.; Williams, A. Interferon-γ increases neuronal death in response to amyloid-β1-42. J. Neuro-inflammation 2006, 3, 7.
8. Baumgart, M.; Moos, V.; Schuhbauer, D.; Müller, B. Differential expression of major histocompatibility complex class II genes on murine macrophages associated with T cell cytokine profile and protective/suppressive effects. Proc. Natl. Acad. Sci. U.S.A. 1998, 95, 6936-6940.
9. Bhowmick, R., et al., Abstract 3133: Reprogramming of tumor-associated macrophages by a short synthetic peptide eradicates ovarian cancer. Cancer Research, 2018. 78(13 Supplement): p. 3133-3133.
10. Biswas, S. K.; Mantovani, A. Macrophage plasticity and interaction with lymphocyte subsets: cancer as a paradigm. Nat. Immunol. 2010, 11, 889.
11. Brown, J. M.; Recht, L.; Strober, S. The Promise of Targeting Macrophages in Cancer Therapy. Clin. Cancer Res. 2017, 23, 3241-3250.
12. Chang, Y.-C.; Chen, T.-C.; Lee, C.-T.; Yang, C.-Y.; Wang, H.-W.; Wang, C.-C.; Hsieh, S.-L. Epigenetic control of MHC class II expression in tumor-associated macrophages by decoy receptor 3. Blood 2008, 111, 5054-5063.
13. Cheng, H.; Wang, Z.; Fu, L.; Xu, T. Macrophage Polarization in the Development and Progression of Ovarian Cancers: An Overview. Front. Oncol. 2019, 9, 421.
14. Cheng, L.; Wang, Y.; Huang, L. Exosomes from M1-Polarized Macrophages Potentiate the Cancer Vaccine by Creating a Pro-inflammatory Microenvironment in the Lymph Node. Mol. Ther. 2017, 25, 1665-1675.
15. Chhor, V., et al., Characterization of phenotype markers and neuronotoxic potential of polarised primary microglia in vitro. Brain, behavior, and immunity, 2013. 32: p. 70-85.
16. Choi, D.-S.; Kim, D.-K.; Kim, Y.-K.; Gho, Y. S. Proteomics of extracellular vesicles: Exosomes and ectosomes. Mass Spectrom. Rev. 2015, 34, 474-490.
17. Choo, Y. W.; Kang, M.; Kim, H. Y.; Han, J.; Kang, S.; Lee, J. R.; Jeong, G. J.; Kwon, S. P.; Song, S. Y.; Go, S.; Jung, M.; Hong, J.; Kim, B. S. M1 Macrophage-Derived Nanovesicles Potentiate the Anticancer Efficacy of Immune Checkpoint Inhibitors. ACS Nano 2018, 12, 8977-8993.
18. Chun, B. J., et al., Simulation of P2X-mediated calcium signalling in microglia. J Physiol, 2019. 597(3): p. 799-818.
19. Conroy, S. M.; Nguyen, V.; Quina, L. A.; Blakely-Gonzales, P.; Ur, C.; Netzeband, J. G.; Prieto, A. L.; Gruol, D. L. Interleukin-6 produces neuronal loss in developing cerebellar granule neuron cultures. J. Neuroimmunol. 2004, 155, 43-54.
20. David, S.; Kroner, A. Repertoire of microglial and macrophage responses after spinal cord injury. Nat. Rev. Neurosci. 2011, 12, 388.
21. De Camilli, P.; Takei, K.; McPherson, P. S. The function of dynamin in endocytosis. Curr. Opin. Neurobiol. 1995, 5, 559-565.
22. Deming, D. A., et al., A phase I study of selumetinib (AZD6244/ARRY-142866), a MEK1/2 inhibitor, in combination with cetuximab in refractory solid tumors and KRAS mutant colorectal cancer. Invest New Drugs, 2016. 34(2): p. 168-75.
23. Dirkx, A. E., et al., Monocyte/macrophage infiltration in tumors: modulators of angiogenesis. J Leukoc Biol, 2006. 80(6): p. 1183-96.
24. Dong, X.; Gao, J.; Zhang, C. Y.; Hayworth, C.; Frank, M.; Wang, Z. Neutrophil Membrane-Derived Nanovesicles Alleviate Inflammation To Protect Mouse Brain Injury from Ischemic Stroke. ACS Nano 2019, 13, 1272-1283.
25. Doyle, L.; Wang, M. Overview of Extracellular Vesicles, Their Origin, Composition, Purpose, and Methods for Exosome Isolation and Analysis. Cells 2019, 8, 727.
26. Duque, G. A.; Descoteaux, A. Macrophage cytokines: involvement in immunity and infectious diseases. Front. Immunol. 2014, 5, 491.
27. Elhelu, M. The role of macrophages in immunology. J. Natl. Med. Assoc. 1983, 75, 314-317.
28. Endres, R. G., Signaling crosstalk: new insights require new vocabulary. Biophysical Journal, 2012. 103(11): p. 2241-2242.
29. Fan, Q. M., et al., Tumor-associated macrophages promote cancer stem cell-like properties via transforming growth factor-beta1-induced epithelial-mesenchymal transition in hepatocellular carcinoma. Cancer Lett, 2014. 352(2): p. 160-8.
30. Fang, X.; Duan, Y.; Adkins, G. B.; Pan, S.; Wang, H.; Liu, Y.; Thong, W. Highly Efficient Exosome Isolation and Protein Analysis by an Integrated Nanomaterial-Based Platform. Anal. Chem. 2018, 90, 2787-2795.
31. Feng, D.; Zhao, W.-L.; Ye, Y.-Y.; Bai, X.-C.; Liu, R.-Q.; Chang, L.-F.; Zhou, Q.; Sui, S.-F. Cellular internalization of exosomes occurs through phagocytosis. Traffic 2010, 11, 675-687.
32. Franco, R. and D. Fernandez-Suarez, Alternatively activated microglia and macrophages in the central nervous system. Prog Neurobiol, 2015. 131: p. 65-86.
33. Gao, J.; Chu, D.; Wang, Z. Cell membrane-formed nanovesicles for disease-targeted delivery. J. Controlled Release 2016, 224, 208-216.
34. Gao, J.; Wang, S.; Wang, Z. High yield, scalable and remotely drug-loaded neutrophil-derived extracellular vesicles (EVs) for anti-inflammation therapy. Biomaterials 2017, 135, 62-73.
35. Gensel, J. C.; Kopper, T. J.; Zhang, B.; Orr, M. B.; Bailey, W. M. Predictive screening of M1 and M2 macrophages reveals the immunomodulatory effectiveness of post spinal cord injury azithromycin treatment. Sci. Rep. 2017, 7, 40144.
36. Gensel, J. C.; Nakamura, S.; Guan, Z.; van Rooijen, N.; Ankeny, D. P.; Popovich, P. G. Macrophages Promote Axon Regeneration with Concurrent Neurotoxicity. J. Neurosci. 2009, 29, 3956-3968.
37. Gensel, J. C.; Wang, Y.; Guan, Z.; Beckwith, K. A.; Braun, K. J.; Wei, P.; McTigue, D. M.; Popovich, P. G. Toll-Like Receptors and Dectin-1, a C-Type Lectin Receptor, Trigger Divergent Functions in CNS Macrophages. J. Neurosci. 2015, 35, 9966-9976.
38. Gensel, J. C.; Zhang, B. Macrophage activation and its role in repair and pathology after spinal cord injury. Brain Res. 2015, 1619, 1-11.
39. Gharib, S. A., et al., Transcriptional and functional diversity of human macrophage repolarization. J Allergy Clin Immunol, 2019. 143(4): p. 1536-1548.
40. Goh, W. J.; Zou, S.; Ong, W. Y.; Torta, F.; Alexandra, A. F.; Schiffelers, R. M.; Storm, G.; Wang, J.-W.; Czarny, B.; Pastorin, G. Bioinspired Cell-Derived Nanovesicles versus Exosomes as Drug Delivery Systems: a Cost-Effective Alternative. Sci. Rep. 2017, 7, 14322.
41. Gold, E. S.; Underhill, D. M.; Morrissette, N. S.; Guo, J.; McNiven, M. A.; Aderem, A. Dynamin 2 is required for phagocytosis in macrophages. J. Exp. Med. 1999, 190, 1849-1856.
42. Gottlieb, R. A.; Adachi, S. Nitrogen Cavitation for Cell Disruption to Obtain Mitochondria from Cultured Cells. Apoptosis 2000, 213-221.
43. Green, A. E.; Rose, P. G. Pegylated liposomal doxorubicin in ovarian cancer. Int. J. Nanomed. 2006, 1, 229-239.
44. Greenhalgh, A. D.; David, S. Differences in the Phagocytic Response of Microglia and Peripheral Macrophages after Spinal Cord Injury and Its Effects on Cell Death. J. Neurosci. 2014, 34, 6316-6322.
45. Gu, Z.; Biswas, A.; Zhao, M.; Tang, Y. Tailoring nanocarriers for intracellular protein delivery. Chem. Soc. Rev. 2011, 40, 3638-55.
46. György, B., et al., Therapeutic applications of extracellular vesicles: Clinical promise and open questions, in Annual Review of Pharmacology and Toxicology. 2015. p. 439-464.
47. Ha, D.; Yang, N.; Nadithe, V. Exosomes as therapeutic drug carriers and delivery vehicles across biological membranes: current perspectives and future challenges. Acta Pharm Sin B 2016, 6, 287-96.
48. Hahn, K. K., J. J. Wolff, and J. M. Kolesar, Pharmacogenetics and irinotecan therapy. Am J Health Syst Pharm, 2006. 63(22): p. 2211-7.
49. Han, X., et al., CD47, a Ligand for the Macrophage Fusion Receptor, Participates in Macrophage Multinucleation. 2000. 275(48): p. 37984-37992.
50. Hirayama, D.; Iida, T.; Nakase, H. The Phagocytic Function of Macrophage-Enforcing Innate Immunity and Tissue Homeostasis. Int. J. Mol. Sci. 2017, 19, 92.
51. Hoffmann, A., et al., The IkappaB-NF-kappaB signaling module: temporal control and selective gene activation. Science, 2002. 298(5596): p. 1241-5.
52. Hon, K. W.; Abu, N.; Ab Mutalib, N.-S.; Jamal, R. Exosomes As Potential Biomarkers and Targeted Therapy in Colorectal Cancer: A Mini-Review. Front. Pharmacol. 2017, 8, 583.
53. Jang, S. C.; Kim, O. Y.; Yoon, C. M.; Choi, D.-S.; Roh, T.-Y.; Park, J.; Nilsson, J.; Lotvall, J.; Kim, Y.-K.; Gho, Y. S. Bioinspired exosome-mimetic nanovesicles for targeted delivery of chemo-therapeutics to malignant tumors. ACS Nano 2013, 7, 7698.
54. Jeong, D.; Jo, W.; Yoon, J.; Kim, J.; Gianchandani, S.; Gho, Y. S.; Park, J. Nanovesicles engineered from ES cells for enhanced cell proliferation. Biomaterials 2014, 35, 9302-10.
55. Jiang, X.-C. and J.-Q. Gao, Exosomes as novel bio-carriers for gene and drug delivery. International Journal of Pharmaceutics, 2017. 521(1): p. 167-175.
56. Jo, W., et al., Microfluidic fabrication of cell-derived nanovesicles as endogenous RNA carriers. Lab Chip, 2014. 14(7): p. 1261-9.
57. Jo, W., et al., Large-scale generation of cell-derived nanovesicles. Nanoscale, 2014. 6(20): p. 12056-64.
58. Kanazawa, M.; Ninomiya, I.; Hatakeyama, M.; Takahashi, T.; Shimohata, T. Microglia and Monocytes/Macrophages Polarization Reveal Novel Therapeutic Mechanism against Stroke. Int. J. Mol. Sci. 2017, 18, 2135.
59. Kekenes-Huskey, P. M., et al., Molecular and subcellular-scale modeling of nucleotide diffusion in the cardiac myofilament lattice. Biophys J, 2013. 105(9): p. 2130-40.
60. Kigerl, K. A.; Gensel, J. C.; Ankeny, D. P.; Alexander, J. K.; Donnelly, D. J.; Popovich, P. G. Identification of two distinct macrophage subsets with divergent effects causing either neurotoxicity or regeneration in the injured mouse spinal cord. J. Neurosci. 2009, 29, 13435-13444.
61. Kim, H.; Wang, S. Y.; Kwak, G.; Yang, Y.; Kwon, I. C.; Kim, S. H. Exosome-Guided Phenotypic Switch of M1 to M2 Macrophages for Cutaneous Wound Healing. Adv. Sci. 2019, 6, 1900513.
62. Kumar, A.; Dixit, C. K., 3—Methods for characterization of nanoparticles; Elsevier Ltd, 2017; pp 43-58.
63. Laird, M. H., et al., TLR4/MyD88/PI3K interactions regulate TLR4 signaling. J Leukoc Biol, 2009. 85(6): p. 966-77.
64. Lakhal, S. and M. J. Wood, Exosome nanotechnology: An emerging paradigm shift in drug delivery: Exploitation of exosome nanovesicles for systemic in vivo delivery of RNAi heralds new horizons for drug delivery across biological barriers. BioEssays, 2011. 33(10):p. 737-741.
65. Lässer, C., S. C. Jang, and J. Lötvall, Subpopulations of extracellular vesicles and their therapeutic potential. Molecular Aspects of Medicine, 2018. 60: p. 1-14.
66. Lawrence, T. and G. Natoli, Transcriptional regulation of macrophage polarization: enabling diversity with identity. Nat Rev Immunol, 2011. 11(11): p. 750-61.
67. Levin, M.C., et al., Evaluation of macrophage-specific promoters using lentiviral delivery in mice. Gene therapy, 2012. 19(11): p. 1041-1047.
68. Li, S. P.; Lin, Z. X.; Jiang, X. Y.; Yu, X. Y. Exosomal cargo-loading and synthetic exosome-mimics as potential therapeutic tools. Acta Pharmacol. Sin. 2018, 39, 542-551.
69. Lin, Y.; Xu, J.; Lan, H. Tumor-associated macrophages in tumor metastasis: biological roles and clinical therapeutic applications. J. Hematol. Oncol. 2019, 12, 76.
70. Lindauer, A., et al., Translational Pharmacokinetic/Pharmacodynamic Modeling of Tumor Growth Inhibition Supports Dose-Range Selection of the Anti-PD-1 Antibody Pembrolizumab. CPT Pharmacometrics Syst Pharmacol, 2017. 6(1): p. 11-20.
71. Mantovani, A.; Biswas, S. K.; Galdiero, M. R.; Sica, A.; Locati, M. Macrophage plasticity and polarization in tissue repair and remodelling. J. Pathol. 2013, 229, 176-185.
72. Mantovani, A.; Marchesi, F.; Malesci, A.; Laghi, L.; Allavena, P. Tumour-associated macrophages as treatment targets in oncology. Nat. Rev. Clin. Oncol. 2017, 14, 399-416.
73. Martinez, F. O., et al., Macrophage activation and polarization. Front Biosci, 2008. 13: p. 453-61.
74. Meckes, D. G., et al., Human tumor virus utilizes exosomes for intercellular communication. Proceedings of the National Academy of Sciences, 2010. 107(47): p. 20370.
75. Midekessa, G.; Godakumara, K.; Ord, J.; Viil, J.; Lättekivi, F.; Dissanayake, K.; Kopanchuk, S.; Rinken, A.; Andronowska, A.; Bhattacharjee, S.; Rinken, T.; Fazeli, A. Zeta potential of extracellular vesicles: toward understanding the attributes that determine colloidal stability. ACS Omega 2020, 5, 16701-16710.
76. Mills, C. D.; Kincaid, K.; Alt, J. M.; Heilman, M. J.; Hill, A. M. M-1/M-2 macrophages and the Th1/Th2 paradigm. J. Immunol. 2000, 164, 6166-6173.
77. Milosevits, G., J. Szebeni, and S. Krol, Exosomes: potential model for complement-stealth delivery systems, in European Journal of Nanomedicine. 2015. p. 207.
78. Mitra, K., et al., Integrative approaches for finding modular structure in biological networks. Nat Rev Genet, 2013. 14(10): p. 719-32.
79. Mitragotri, S.; Burke, P. A.; Langer, R. Overcoming the challenges in administering biopharmaceuticals: formulation and delivery strategies. Nat. Rev. Drug Discovery 2014, 13, 655-72.
80. Moonschi, F. H.; Effinger, A. K.; Zhang, X.; Martin, W. E.; Fox, A. M.; Heidary, D. K; DeRouchey, J. E.; Richards, C. I. Cell-Derived Vesicles for Single-Molecule Imaging of Membrane Proteins. Angew Chem Int Ed Engl 2015, 31, 201408707.
81. Moonschi, F. H.; Hughes, C. B.; Mussman, G. M.; Fowlkes, J. L.; Richards, C. I.; Popescu, I. Advances in micro- and nanotechnologies for the GLP-1-based therapy and imaging of pancreatic beta-cells. Acta diabetologica 2018, 55, 405-418.
82. Morgan, R. J., Jr., et al., Ovarian Cancer, Version 1.2016, NCCN Clinical Practice Guidelines in Oncology. J Natl Compr Canc Netw, 2016. 14(9): p. 1134-63.
83. Mout, R.; Ray, M.; Tay, T.; Sasaki, K.; Yesilbag Tonga, G.; Rotello, V. M. General Strategy for Direct Cytosolic Protein DeliveryviaProtein-Nanoparticle Co-engineering. ACS Nano 2017, 11, 6416-6421.
84. Murray, Peter J., et al., Macrophage Activation and Polarization: Nomenclature and Experimental Guidelines. Immunity, 2014. 41(1): p. 14-20.
85. Nakano, T., et al., Uremic Toxin Indoxyl Sulfate Promotes Proinflammatory Macrophage Activation Via the Interplay of OATP2B1 and D114-Notch Signaling. Circulation, 2019. 139(1): p. 78-96.
86. Neijt, J. P., et al., Exploratory phase III study of paclitaxel and cisplatin versus paclitaxel and carboplatin in advanced ovarian cancer. J Clin Oncol, 2000. 18(17): p. 3084-92.
87. Novak, M. L.; Weinheimer-Haus, E. M.; Koh, T. J. Macrophage activation and skeletal muscle healing following traumatic injury. J. Pathol. 2014, 232, 344-355.
88. Noy, R. and J. W. Pollard, Tumor-associated macrophages: from mechanisms to therapy. Immunity, 2014. 41(1): p. 49-61.
89. Ohsawa, K., et al., Microglia/macrophage-specific protein Ibal binds to fimbrin and enhances its actin-bundling activity. J Neurochem, 2004. 88(4): p. 844-56.
90. Pai-Scherf, L., et al., FDA Approval Summary: Pembrolizumab for Treatment of Metastatic Non-Small Cell Lung Cancer: First-Line Therapy and Beyond. Oncologist, 2017. 22(11): p. 1392-1399.
91. Park, K. M.; Bowers, W. J. Tumor necrosis factor-alpha mediated signaling in neuronal homeostasis and dysfunction. Cell. Signal. 2010, 22, 977-983.
92. Patel, S. G.; Sayers, E. J.; He, L.; Narayan, R.; Williams, T. L.; Mills, E. M.; Allemann, R. K.; Luk, L. Y. P.; Jones, A. T.; Tsai, Y. H. Cell-penetrating peptide sequence and modification dependent uptake and subcellular distribution of green florescent protein in different cell lines. Sci. Rep. 2019, 9, 6298.
93. Perry, V. H.; Teeling, J. Microglia and macrophages of the central nervous system: the contribution of microglia priming and systemic inflammation to chronic neurodegeneration. Semin. Immunopathol. 2013, 35, 601-612.
94. Pitt, J. M.; André, F.; Amigorena, S.; Soria, J.-C.; Eggermont, A.; Kroemer, G.; Zitvogel, L. Dendritic cell-derived exosomes for cancer therapy. J. Clin. Investig 2016, 126, 1224-1232.
95. Ramanathan, S.; Jagannathan, N. Tumor associated macrophage: a review on the phenotypes, traits and functions. Iran. J. Cancer Prev. 2014, 7, 1-8.
96. Rana, S., et al., Toward tailored exosomes: the exosomal tetraspanin web contributes to target cell selection. Int J Biochem Cell Biol, 2012. 44(9): p. 1574-84.
97. Rappoport, J. Z.; Heyman, K. P.; Kemal, S.; Simon, S. M. Dynamics of Dynamin during Clathrin Mediated Endocytosis in PC12 Cells (Dynamin during Endocytosis). PLoS One 2008, 3, No. e2416.
98. Robbins, P. D. and A. E. Morelli, Regulation of immune responses by extracellular vesicles. Nat Rev Immunol, 2014. 14(3): p. 195-208.
99. Ruffell, B. and L. M. Coussens, Macrophages and therapeutic resistance in cancer. Cancer Cell, 2015. 27(4): p. 462-72.
100. Pannu, K. K., E. T. Joe, and S. B. Iyer, Performance evaluation of QuantiBRITE phycoerythrin beads. Cytometry, 2001. 45(4): p. 250-8.
101. Petty, A. J. and Y. Yang, Tumor-associated macrophages: implications in cancer immunotherapy. Immunotherapy, 2017. 9(3): p. 289-302.
102. Pospichalova, V., et al., Simplified protocol for flow cytometry analysis of fluorescently labeled exosomes and microvesicles using dedicated flow cytometer. Journal of extracellular vesicles, 2015. 4: p. 25530-25530.
103. Ryall, K. A. and A. C. Tan, Systems biology approaches for advancing the discovery of effective drug combinations. J Cheminform, 2015. 7: p. 7.
104. Sandin, J. N.; Aryal, S. P.; Wilkop, T.; Richards, C. I.; Grady, M. E. Near Simultaneous Laser Scanning Confocal and Atomic Force Microscopy (Conpokal) on Live Cells. JoVE 2020, 11, 162.
105. Sercombe, L.; Veerati, T.; Moheimani, F.; Wu, S. Y.; Sood, A. K.; Hua, S. Advances and Challenges of Liposome Assisted Drug Delivery. Front. Pharmacol. 2015, 6, 286.
106. Sgadari, C.; Angiolillo, A.; Tosato, G. Inhibition of Angio-genesis by Interleukin-12 Is Mediated by the Interferon-Inducible Protein 10. Blood 1996, 87, 3877-3882.
107. Sharma, A.; S. Sharma, U. Liposomes in drug delivery: Progress and limitations. Int. J. Pharm. 1997, 154, 123-140.
108. Shenoda, B. B. and S. K. Ajit, Modulation of Immune Responses by Exosomes Derived from Antigen-Presenting Cells. Clinical medicine insights. Pathology, 2016. 9(Suppl 1): p. 1-8.
109. Shimizu, T., et al., Organic cation transporter Octnl-mediated uptake of food-derived antioxidant ergothioneine into infiltrating macrophages during intestinal inflammation in mice. Drug Metab Pharmacokinet, 2015. 30(3): p. 231-9.
110. Sica, A.; Mantovani, A. Macrophage plasticity and polarization: in vivo veritas. J. Clin. Invest. 2012, 122, 787-795.
111. Siegel, R. L., K. D. Miller, and A. Jemal, Cancer Statistics, 2019. CA Cancer J Clin, 2019. 69(1): p. 7-34.
112. Simpson, R. J. Disruption of cultured cells by nitrogen cavitation. Cold Spring Harbor protocols 2010, 2010, pdb-prot5513.
113. Singh, G., et al., Macrophage Gene Therapy: opening novel therapeutic avenues for immune disorders. 2015.
114. Snell, A. A.; Neupane, K. R.; McCorkle, J. R.; Fu, X.; Moonschi, F. H.; Caudill, E. B.; Kolesar, J.; Richards, C. I. Cell-Derived Vesicles for in Vitro and in Vivo Targeted Therapeutic Delivery. ACS Omega 2019, 4, 12657-12664.
115. Sorensen, E. W.; Gerber, S. A.; Frelinger, J. G.; Lord, E. M. IL-12 suppresses vascular endothelial growth factor receptor 3 expression on tumor vessels by two distinct IFN-γ-dependent mechanisms. J. Immunol. 2010, 184, 1858-1866.
116. Starchenko, A. and D. A. Lauffenburger, In vivo systems biology approaches to chronic immune/inflammatory pathophysiology. Curr Opin Biotechnol, 2018. 52: p. 9-16.
117. Stewart, M. P.; Sharei, A.; Ding, X.; Sahay, G.; Langer, R.; Jensen, K. F. In vitro and ex vivo strategies for intracellular delivery. Nature 2016, 538, 183-192.
118. Stewart, B. D., et al., Computational modeling of amylin-induced calcium dysregulation in rat ventricular cardiomyocytes. Cell Calcium, 2018. 71: p. 65-74.
119. Sťimac, A.; Tokić, M.; Ljubetič, A.; Vuletid, T.; Sěkutor, M.; Pozar, J.; Leko, K.; Hanževacki, M.; Frkanec, L.; Frkanec, R. Functional self-assembled nanovesicles based on β-cyclodextrin, liposomes and adamantyl guanidines as potential nonviral gene delivery vectors. Org. Biomol. Chem. 2019, 17, 4640.
120. Stout, R. D.; Suttles, J. Functional plasticity of macrophages: reversible adaptation to changing microenvironments. J. Leukocyte Biol. 2004, 76, 509-513.
121. Sun, L.; He, C.; Nair, L.; Yeung, J.; Egwuagu, C. E. Interleukin 12 (IL-12) family cytokines: Role in immune pathogenesis and treatment of CNS autoimmune disease. Cytokine 2015, 75, 249-255.
122. Sundborger, A. C.; Hinshaw, J. E. Regulating dynamin dynamics during endocytosis. F1000Prime Rep. 2014, 6, 85.
123. Tan, A., H. De La Peña, and A. M. Seifalian, The application of exosomes as a nanoscale cancer vaccine. International journal of nanomedicine, 2010. 5: p. 889-900.
124. Tan, X.; Zhang, Y.; Wang, Q.; Ren, T.; Gou, J.; Guo, W.; Yin, T.; He, H.; Zhang, Y.; Tang, X. Cell-penetrating peptide together with PEG-modified mesostructured silica nanoparticles promotes mucous permeation and oral delivery of therapeutic proteins and peptides. Biomater. Sci. 2019, 7, 2934-2950.
125. Tang, X., et al., Anti-tumour strategies aiming to target tumour-associated macrophages. Immunology, 2013. 138(2): p. 93-104.
126. Tariq, M.; Zhang, J.; Liang, G.; Ding, L.; He, Q.; Yang, B. Macrophage Polarization: Anti-Cancer Strategies to Target Tumor-Associated Macrophage in Breast Cancer. J. Cell. Biochem. 2017, 118, 2484-2501.
127. Tariq, M., et al., Gefitinib inhibits M2-like polarization of tumor-associated macrophages in Lewis lung cancer by targeting the STAT6 signaling pathway. Acta Pharmacol Sin, 2017. 38(11): p. 1501-1511.
128. Vasilyev, F. F., J. A. Lopatnikova, and S. V. Sennikov, Optimized flow cytometry protocol for analysis of surface expression of interleukin-1 receptor types I and II. Cytotechnology, 2013. 65(5): p. 795-802.
129. Taylor, D. D.; Shah, S. Methods of isolating extracellular vesicles impact down-stream analyses of their cargoes. Methods 2015, 87, 3-10.
130. Tippett, E., et al., Characterization of tetraspanins CD9, CD53, CD63, and CD81 in monocytes and macrophages in HIV-1 infection. Journal of Leukocyte Biology, 2013. 93(6): p. 913-920.
131. Wang, L., et al., Comparison of fluorescein and phycoerythrin conjugates for quantifying CD20 expression on normal and leukemic B-cells. Cytometry B Clin Cytom, 2006. 70(6): p. 410-5.
132. Wang, N., H. Liang, and K. Zen, Molecular mechanisms that influence the macrophage ml-m2 polarization balance. Frontiers in immunology, 2014. 5: p. 614-614.
133. Wang, Z.; Ling, L.; Du, Y.; Yao, C.; Li, X. Reduction responsive liposomes based on paclitaxel-ss-lysophospholipid with high drug loading for intracellular delivery. Int. J. Pharm. 2019, 564, 244.
134. Winslow, R. L., et al., Computational Medicine: Translating Models to Clinical Care. 2012. 4(158): p. 158rva1-158rv11.
135. Wu, T.; Qi, Y.; Zhang, D.; Song, Q.; Yang, C.; Hu, X.; Bao, Y.; Zhao, Y.; Zhang, Z. Bone Marrow Dendritic Cells Derived Microvesicles for Combinational Immunochemotherapy against Tumor. Adv. Funct. Mater. 2017, 27, 1703191.
136. Xiong, Q.; Lee, G. Y.; Ding, J.; Li, W.; Shi, J. Biomedical applications of mRNA nanomedicine. Nano Res. 2018, 11, 5281-5309.
137. Yin, W., et al., Remodeling Tumor-Associated Macrophages and Neovascularization Overcomes EGFR(T790M) -Associated Drug Resistance by PD-L1 Nanobody-Mediated Codelivery. Small, 2018. 14(47): p. e1802372.
138. You, Q.; Gong, Q.; Han, Y. Q.; Pi, R.; Du, Y. J.; Dong, S. Z. Role of miR-124 in the regulation of retinoic acid-induced Neuro-2A cell differentiation. Neural Regen. Res. 2020, 15, 1133-1139.
139. Yurkin, S. T.; Wang, Z. Cell membrane-derived nanoparticles: emerging clinical opportunities for targeted drug delivery. Nano-medicine 2017, 12, 2007-2019.
140. Thai, Y.; Su, J.; Ran, W.; Zhang, P.; Yin, Q.; Zhang, Z.; Yu, H.; Li, Y. Preparation and Application of Cell Membrane-Camouflaged Nanoparticles for Cancer Therapy. Theranostics 2017, 7, 2575-2592.
141. Zhang, B.; Bailey, W. M.; Kopper, T. J.; Orr, M. B.; Feola, D. J.; Gensel, J. C. Azithromycin Drives Alternative Macrophage Activation and Improves Recovery and Tissue Sparing in Contusion Spinal Cord Injury. J. Neuroinflammation 2015, 12, 218.
142. Zhang, M., et al., Anti-CD47 Treatment Stimulates Phagocytosis of Glioblastoma by M1 and M2 Polarized Macrophages and Promotes M1 Polarized Macrophages In Vivo. PLoS One, 2016. 11(4): p. e0153550.
143. Zhang, B.; Kopper, T. J.; Liu, X.; Cui, Z.; Van Lanen, S. G.; Gensel, J. C. Macrolide Derivatives Reduce Proinflammatory Macrophage Activation and Macrophage-Mediated Neurotoxicity. CNS Neurosci. Ther. 2019, 25, 591-600.
144. Zhou, T. Y., et al., Interleukin-6 induced by YAP in hepatocellular carcinoma cells recruits tumor-associated macrophages. J Pharmacol Sci, 2018. 138(2): p. 89-95.
145. Zhu, L.; Oh, J. M.; Gangadaran, P.; Kalimuthu, S.; Baek, S. H.; Jeong, S. Y.; Lee, S. W.; Lee, J.; Ahn, B. C. Targeting and therapy of glioblastoma in a mouse model using exosomes derived from Natural Killer cells. Front. Pharmacol. 2018, 9, 824.
146. Zhu, J.-Y.; Zheng, D.-W.; Zhang, M.-K.; Yu, W.-Y.; Qiu, W.-X.; Hu, J.-J.; Feng, J.; Zhang, X.-Z. Preferential Cancer Cell Self-Recognition and Tumor Self-Targeting by Coating Nanoparticles with Homotypic Cancer Cell Membranes. Nano Lett. 2016, 16, 5895-5901.
147. Zhu, Y., et al., CSF1/CSF1R blockade reprograms tumor-infiltrating macrophages and improves response to T-cell checkpoint immunotherapy in pancreatic cancer models. Cancer Res, 2014. 74(18): p. 5057-69.

It will be understood that various details of the presently disclosed subject matter can be changed without departing from the scope of the subject matter disclosed herein. Furthermore, the foregoing description is for the purpose of illustration only, and not for the purpose of limitation.

1. A method of making a macrophage-derived engineered vesicle (MEV), comprising:
(a) providing a first macrophage of a first phenotype; and
(b) fragmenting a cell membrane of the first macrophage and allowing the fragmented membrane to assemble into a first phenotype MEV derived from the first macrophage.

2. The method of claim 1, and further comprising suspending the cell membrane fragments of the first macrophage in an assembly solution comprising cargo such that the first phenotype MEV derived from the first macrophage encapsulates the cargo during assembly.

3. The method of claim 1, and further comprising incubating the first phenotype MEV derived from the first macrophage with a second macrophage of a second phenotype, thereby shifting the phenotype of the second macrophage to the first phenotype.

4. A method of modifying a phenotype of a macrophage, comprising:
(a) providing a macrophage of a second phenotype; and
(b) incubating the macrophage with a macrophage-derived engineered vesicle (MEV) of a first phenotype, thereby shifting the macrophage to a first phenotype.

5. The method of claim 4, wherein the macrophage is obtained from a target environment.

6. The method of claim 5, wherein the target environment is an in vitro target environment or an in vivo target environment.

7. The method of claim 4, fragmenting a cell membrane of the macrophage that has shifted to a first phenotype, and allowing the fragmented membrane to assemble into a first phenotype MEV.

8. The method of claim 7, and further comprising suspending the cell membrane fragments of the first macrophage in an assembly solution comprising cargo such that the first phenotype MEV derived from the first macrophage encapsulates the cargo during assembly.

9. The method of claim 5, and further comprising contacting the first phenotype MEV with the target environment.

10. The method of claim 9, wherein the target environment is an in vivo environment.

11. The method of claim 10, wherein the in vivo environment is the site of a condition in a subject.

12. The method of claim 11, wherein the condition is selected from the group consisting of a cancer, a condition of the central nervous system, a wound, an inflammatory disease, an infectious disease, a traumatic injury, and an ischemic event.

13. The method of claim 12, wherein the condition is a cancer, and the in vivo environment is the cancer or tumor micro environment.

14. The method of claim 1, wherein the first phenotype is selected from the group consisting of M0, M1, and M2.

15. The method of claim 3, wherein the second phenotype is selected from the group consisting of M0, M1, and M2, so long as the second phenotype is distinct from the first phenotype.

16. The method of claim 2, wherein cargo is encapsulated by the MEV, and the cargo is selected from the group consisting of genetic material, therapeutic agent, protein, and fluorescent marker.

17. The method of claim 1, wherein the macrophage is obtained from human peripheral blood mononuclear cell-derived monocytes or bone marrow.

18. The method of claim 17, and further comprising stimulating the macrophages with lipopolysaccharide (LPS) and interferon gamma (IFN-γ).

19. The method of claim 17, and further comprising stimulating the macrophages with interleukin 4 (IL-4) and/or interleukin-13 (IL-13).

20. A composition, comprising a macrophage-derived engineered vesicle (MEV) having a first phenotype, derived from a target environment, and optionally encapsulating cargo.
